(19) United States
(12) Patent Application Publication
(10) Pub. No.: US 2024/0366894 A1
(43) Pub. Date: Nov. 7, 2024

(54) METHODS FOR PROVIDING RESPIRATORY SUPPORT WITH CUFF DEFLATION

(71) Applicant: Fisher & Paykel Healthcare Limited, Auckland (NZ)

(72) Inventors: Matthew Jon PAYTON, Auckland (NZ); Anthony Behram ZEMKE, Auckland (NZ); Alicia Jerram Hunter EVANS, Auckland (NZ); Craig Karl WHITE, Auckland (NZ); Anil PATEL, Auckland (NZ); Thomas Heinrich BARNES, Auckland (NZ)

(21) Appl. No.: 18/557,863

(22) PCT Filed: Apr. 29, 2022
(86) PCT No.: PCT/IB2022/053979

§ 371 Date: Oct. 27, 2023

Related U.S. Application Data
(60) Provisional application No. 63/201,438, filed on Apr. 29, 2021.

(30) Foreign Application Priority Data
Aug. 24, 2021 (AU) 2021221513

(51) Int. Cl. A61M 16/04; A61M 16/00; A61M 16/01; A61M 16/08

(52) U.S. Cl. CPC A61M 16/045; A61M 16/01; A61M 16/022; A61M 16/0465; A61M 16/0816

(57) ABSTRACT

A method for providing respiratory support to a patient includes intubating the patient with an invasive patient interface having a sealable member operable to form a sealing engagement with the patient's airway. While the patient is intubated, the sealable member is operated to form a non-sealing arrangement of the invasive patient interface within the patient's airway and a flow of respiratory gas is provided to the patient via the invasive patient interface. This respiratory support is provided during absence of spontaneous breathing.

### TECHNICAL FIELD

The present invention relates to methods and systems for providing respiratory support to a patient. It relates particularly, but not exclusively, to methods and systems for providing respiratory support to the patient through an invasive patient interface, where the form of respiratory support may be selected based upon a sealing engagement of the invasive patient interface with the patient's airway, or a non-sealing arrangement of the invasive patient interface within the patient's airway.

### BACKGROUND OF INVENTION

Patients often require a form of respiratory support during medical procedures, particularly medical procedures which involve sedation or anaesthesia. In some cases, patients under sedation or anaesthesia may not be spontaneously breathing and they may be provided respiratory support such as mechanical ventilation to maintain cycles of lung inflation and deflation. Mechanical ventilation may involve delivering a pressure waveform or tidal volume to a patient which may be intended to mimic a cycle of spontaneous breathing.

Ventilation of an apnoeic patient may be provided through an invasive patient interface such as an endotracheal tube (ETT) connected to a ventilator or anaesthesia machine. An ETT is typically kept in place by a cuff at the end of the tube which is inflated to create a seal with the patient's airway. Further examples of invasive patient interfaces that may be used for ventilation include a laryngeal mask airway, and tracheostomy tube.

A potential issue with traditional mechanical ventilation that mimics cycles of spontaneous breathing is that such systems cause physical movement of the patient's diaphragm and lungs with cycles of inflation and deflation. This movement can be undesirable for example during procedures that benefit from stillness of the body. This issue is particularly troublesome in circumstances where a proceduralist wishes to perform a procedure on the patient that requires stillness of the respiratory system and/or abdomen and/or thorax. There is also an issue with ensuring adequate ventilation of the patient throughout such a procedure.

It may be desirable to provide a form of respiratory support that overcomes or at least improves upon one or more of the aforementioned limitations.

A reference herein to a patent document or any other matter identified as prior art, is not to be taken as an admission that the document or other matter was known or that the information it contains was part of the common general knowledge as at the priority date of any of the claims.

### SUMMARY OF INVENTION

Viewed from one aspect, the present disclosure provides a method for providing respiratory support to a patient, the method comprising the steps of: (a) intubating the patient with an invasive patient interface having a sealable member operable to form a sealing engagement with the patient's airway; and (b) while the patient is intubated, operating the sealable member to form a non-sealing arrangement of the invasive patient interface within the patient's airway; and (c) providing a flow of respiratory gas to the patient via the invasive patient interface; wherein the respiratory support is provided during absence of spontaneous breathing.

In some embodiments, the respiratory support increases CO₂ clearance in the patient.

In some embodiments, operating the sealable member to form the non-sealing arrangement provides a first exit flow path between an external wall of the invasive patient interface and the patient's airway for out flow of gases from the patient's airway. Gases in the first exit flow path may have a first predetermined flow rate. The first predetermined flow rate may be at least about 0.5 LPM, preferably at least about 5 LPM or at least about 20 LPM.

In some embodiments, the invasive patient interface provides a second exit flow path for out flow of gases from the patient's airway, in addition to providing a flow of respiratory gas to the patient. Gases in the second exit flow path may have a second predetermined flow rate. The second predetermined flow rate may be less than the first predetermined flow rate.

In some embodiments, the sealable member comprises an inflatable body, and operating the sealable member to form the non-sealing arrangement comprises deflating the inflatable body. In some embodiments, the inflatable body is deflated to a predetermined inflation pressure. The predetermined inflation pressure may be less than a sealing pressure required to achieve sealing engagement with the patient's airway.

In some embodiments, the inflatable body is deflated to achieve a predetermined distance between the inflatable body and the patient's airway. In some embodiments, the inflatable body is deflated to occupy a cross sectional area corresponding to less than 100% occlusion of the region between the invasive patient interface and the patient's airway.

In some embodiments, during deflation of the inflatable body, a parameter may be monitored which is indicative of degree of deflation of the inflatable body. Deflation may be ceased when the parameter reaches a predetermined value. The parameter may be selected from a group including but not limited to: (i) pressure in the inflatable body; (ii) expiratory flow around the invasive patient interface; (iii) expiratory flow within the invasive patient interface; and (iv) velocity of flow out of the invasive patient interface.

In some embodiments, the method may include the step of, prior to intubating the patient, detecting absence of spontaneous breathing by the patient.

In some embodiments, the method includes providing respiratory gas at a therapeutic flow rate. The method may further include controlling a flow rate of respiratory gas provided to the patient. The flow rate may be controlled by an automated control device. The therapeutic flow rate may be in a range of about 0 to about 120 LPM, preferably at least about 20 LPM to about 70 LPM, preferably about 40 LPM. The therapeutic flow rate may be a variable rate. In some embodiments, the flow of respiratory gas may be substantially continuous.

In some embodiments, the method includes providing respiratory gas at a predetermined velocity. The method may further include controlling a velocity of respiratory gas provided to the patient. The predetermined velocity may be in a range of up to about 60 m/s or up to about 30 m/s and preferably at least about 5 m/s. The predetermined velocity may be variable.

In some embodiments, the respiratory support provided via the invasive patient interface achieves a predetermined airway pressure. The predetermined airway pressure may be, for example, about 2 cmH₂O to about 20 cmH₂O, preferably about 2 cmH₂O to about 10 cmH₂O and more preferably about 2 cmH₂O to about 5 cmH₂O.

The invasive patient interface may be selected from a group including but not limited to: (i) an endotracheal tube; (ii) a laryngeal mask airway (LMA); and (iii) a tracheostomy tube.

In some embodiments, the sealable member comprises an inflatable cuff located toward a proximal end of the invasive patient interface which is inflatable to form the sealing engagement with the patient's airway. The non-sealing engagement may be achieved when the inflatable cuff is inflated to an inflation pressure of less than about 30 cmH₂O, preferably less than about 25 cmH₂O or less than about 20 cmH₂O. In some embodiments, the method includes the step of using a pressure sensor to monitor inflation pressure of the inflatable cuff.

In some embodiments, the flow of respiratory gas is provided to the invasive patient interface using a connector having a one or more constrictions. In some embodiments, the one or more constrictions comprise a nozzle having an outflow opening diameter that is smaller than an internal diameter of the invasive patient interface. The nozzle outflow opening may generate pressure within the patient's airway of about 2 cmH₂O to about 20 cmH₂O, preferably about 2 cmH₂O to about 10 cmH₂O and more preferably about 2 cmH₂O to about 5 cmH₂O.

In some embodiments, the method includes, prior to operating the sealable member to form the non-sealing arrangement, inducing the patient into a state of general anaesthesia. Prior to inducing the patient into a state of general anaesthesia, the patient may have been breathing spontaneously. The respiratory support may be provided in the absence of displacement of the patient's diaphragm and/or abdomen and/or thorax. That is, in some embodiments, the method causes little or no movement of the patient. In some embodiments, the respiratory support provided maintains a period of safe apnoea.

The duration of safe apnoea may be defined as the time until a patient reaches a specified oxygen saturation level. Typically, that oxygen saturation level may be 88-92%, although that level may vary depending on the patient and procedure being carried out. Saturations below this level can rapidly deteriorate to critical levels (e.g., <70%) on the steep section of the oxyhaemoglobin dissociation curve posing significant risk to the patient. Alternatively, the duration of safe apnoea may be defined as the time until a patient reaches a or reaches close to a specified CO₂ level, which could be a saturation or threshold level. The duration of safe apnoea can vary considerably patient to patient.

Viewed from another aspect, the present disclosure provides method for providing respiratory support to a patient, comprising the steps of: (a) intubating the patient with an invasive patient interface having a sealable member, wherein the invasive patient interface is operable in a first configuration or a second configuration; (b) providing one form of respiratory support when the invasive patent interface is in one of the first configuration or the second configuration; and (c) responsive to the invasive patient interface being in the other of the first configuration or the second configuration switching to provide a different form of respiratory support.

In some embodiments, in the first configuration the sealing member forms a non-sealing arrangement of the invasive patient interface within the patient's airway, and in the second configuration the sealing member forms a sealing engagement between the invasive patient interface and the patient's airway.

In some embodiments, the form of respiratory support provided when the invasive patent interface is in the first configuration is a substantially continuous flow of breathing gas. The form of respiratory support provided when the invasive patent interface is in the first configuration may be e.g. flow controlled. The flow controlled respiratory support may have a flow rate in a range of about 0 to about 120 LPM, preferably about 20 LPM to about 70 LPM, for example at least about 40 LPM. In some embodiments, the flow controlled respiratory support may have a flow rate which is variable.

In some embodiments, the form of respiratory support provided when the invasive patent interface is in the second configuration comprises a cyclic flow of gas providing inspiratory and expiratory phases. The form of respiratory support provided when the invasive patent interface is in the second configuration may be a pressure and/or volume controlled flow of breathing gas.

In some embodiments, the form of respiratory support provided when the invasive patent interface is in the second configuration may include e.g. mechanical or manual ventilation providing inspiratory and expiratory phases of gas flow within the patient's airway. The mechanical ventilation may be provided by e.g. a ventilator or an anaesthesia machine.

In some embodiments, the form of respiratory support provided when the invasive patent interface is in the second configuration may be dependent on an intended breathing cycle of the patient. Alternatively/additionally, the form of respiratory support provided when the invasive patient interface is in the first configuration is independent of spontaneous or non-spontaneous (i.e. artificially ventilated) inspiratory or expiratory breathing cycles of the patient.

In some embodiments, the one form of respiratory support and the different form of respiratory support are provided by separate flow sources. In some embodiments, when the invasive patient interface is or switched to the first configuration, the method may include coupling the invasive patient interface with a first flow source configured to provide a continuous flow of respiratory gas. In some embodiments, when the invasive patient interface is in or switched to the second configuration, the method may include coupling the invasive patient interface with a second flow source configured to provide a cyclic and/or pressure and/or volume controlled flow of respiratory gas. The method may include, prior to coupling the invasive patient interface with the first flow source or the second flow source, decoupling the invasive patient interface from the other of the first flow source or the second flow source.

In some embodiments, the one form of respiratory support and the different form of respiratory support are provided by a common flow source.

In some embodiments, the method includes humidifying a respiratory gas provided with one or both of the one form of respiratory support and the different form of respiratory support.

In some embodiments, the method may be performed during absence of spontaneous breathing by the patient. The method may further include performing a medical procedure that benefits from little or no movement of the patient due to respiratory gas exchange and, repeating step (c) to achieve the second configuration for performance of the medical procedure.

In some embodiments, the method may include switching or alternating between the one form of respiratory support and the different form of respiratory support. This may involve repeating one or more coupling and decoupling steps, and switching the configuration of the invasive patient interface. Switching between forms of respiratory support may be desirable to achieve one or more of: (a) improving CO2 clearance; (b) providing patient airway pressure; (c) delivering volatile agents to the patient; (d) reducing risk of gastric aspiration; (e) and improving oxygenation.

Viewed from another aspect, the present disclosure provides system for providing respiratory support to a patient during a medical procedure, the system comprising: (a) a flow source operable to provide a flow of gas for delivery to the patient via an invasive patient interface; and (b) a controller receiving one or more inputs and operable to control the flow source according to one or more received inputs; wherein the system is couplable with an invasive patient interface having a sealable member that is operable to achieve a first system condition in which there is non-sealing arrangement of the invasive patient interface within the patient's airway, and a second system condition in which there is sealing engagement with the patient's airway; and wherein, the controller controls the flow source to provide a first respiratory support in the first system condition and a second respiratory support in the second system condition.

In some embodiments, the controller may control the flow source such that the first respiratory support is independent of spontaneous or non-spontaneous (i.e. artificially ventilated) inspiratory or expiratory breathing cycles of the patient, and the second respiratory support is dependent on or imitates an intended breathing cycle of the patient.

In some embodiments, the first respiratory support comprises a substantially continuous flow of gas. The controller may control the rate of flow of gas provided by the flow source. The controller may provide variable control of the rate of flow of gas. In some embodiments, the controller may control the rate of flow of gas in a range of about 0 to about 120 LPM, preferably at least about 40 LPM.

In some embodiments, the second respiratory support may be pressure and/or volume controlled. In some embodiments, the second respiratory support may comprise a cyclic flow of gas providing inspiratory and expiratory phases.

In some embodiments, the controller may control the flow source to provide a flow of gas at a predetermined velocity, preferably in a range of about up to about 60 m/s or up to about 30 m/s and preferably at least about 5 m/s. The controller may provide variable control of the predetermined velocity.

In some embodiments, inputs received by the controller may designate that the system is in the first system condition or the second system condition, and the controller may control the flow source accordingly. For example inputs receivable by the controller may include signals generated by one or more sensors detecting a parameter used to determine if the system is in the first system condition or the second system condition. In some embodiments, inputs receivable by the controller may include signals generated by one or more of, for example, (a) an expiratory gas flow sensor; (b) a pressure sensor associated with an inflatable body comprising the sealable member; (c) an airway pressure sensor; and (d) a user input device. An expiratory gas flow sensor may monitor flows of exiting gases inside the invasive patient interface. Alternatively/additionally, an expiratory gas flow sensor may monitor flows of exiting gases near an external wall of the invasive patient interface.

In some embodiments, the flow source may comprise a first flow source providing a substantially continuous flow of gas, and a second flow source providing a cyclic flow of gas having inspiratory and expiratory phases. Alternatively, the flow source may comprise a single flow source operable to provide: (i) a continuous flow of gas for providing the first respiratory support; and (ii) a cyclic flow of gas having inspiratory and expiratory phases for providing the second respiratory support; wherein the controller controls operation of the flow source to provide the first respiratory support or the second respiratory support.

In some embodiments, the controller may be operable to control one or more of pressure and volume of gas provided by the flow source for delivery to the patient. Alternatively/additionally, the controller may be operable to control the pressure and/or volume substantially in synchrony with inspiratory and expiratory phases of a breathing cycle.

In some embodiments, the system includes a connector couplable with the invasive patient interface for providing the second respiratory support, wherein the connector has one or more constrictions. In some embodiments, the connector may include a nozzle having an outflow opening diameter that is smaller than an internal diameter of the invasive patient interface. The system may include a gas delivery conduit between the flow source and the connector, wherein the connector, when inserted into the invasive patient interface, provides a flow path for delivery of the first respiratory support to the patient. In some embodiments, the connector may be integral with the delivery conduit.

In some embodiments, the system includes means for operating the sealable member to achieve sealing engagement with the patient's airway or a non-sealing engagement of the invasive patient interface within the patient's airway. For example, in embodiments, where the sealable member comprises an inflatable body, the system may include an inflation source for inflating in the inflatable body.

In some embodiments, in order to achieve the second system condition, the controller may control the inflation source to inflate the inflatable body to an inflation pressure sufficient for achieving sealing engagement with the patient's airway, and to achieve the first system condition, the controller may control the inflation source to deflate the inflatable body to a predetermined inflation pressure to achieve non-sealing engagement of the invasive patient interface within the patient's airway. In some embodiments, the predetermined inflation pressure may be less than a sealing pressure required to achieve sealing engagement with the patient's airway.

In some embodiments, in order to achieve the first system condition, the controller may cause deflation of the inflatable body to occupy a cross sectional area corresponding to less than 100% occlusion of the region between the invasive patient interface and the patient's airway.

In some embodiments, the system may include one or more sensors monitoring a parameter used by the controller to determine if the system is in the first system condition or the second system condition. The sensors may include one or more of, for example: (i) a pressure sensor; (ii) a flow sensor; and (iii) a gas flow velocity sensor.

In some embodiments, inputs received by the controller may include a breathing input indicative of spontaneous breathing by the patient, and the controller may control the flow source to provide respiratory support only when the breathing input indicates an absence of spontaneous breathing by the patient.

In some embodiments, inputs received by the controller may include a movement input indicative of movement of the patient during provision of the respiratory support, and the controller may generate a control signal to produce an audible and/or visible and/or haptic output indicating substantial absence of movement during provision of respiratory support by the system. In some embodiments, the system may include a motion sensor configured to detect movement of the patient during provision of the respiratory support.

In some embodiments, the system may include an output means having one or more of a display device, a speaker output module and a haptic feedback module operable by the controller to provide an output discernible by a clinician while performing a medical procedure on the patient.

In some embodiments, the sealable member comprises an inflatable cuff located toward a proximal end of the invasive patient interface which is inflatable to form the sealing engagement with the patient's airway. In some embodiments, in order to achieve the first system condition, the controller controls inflation of the cuff to an inflation pressure of less than about 30 cmH₂O.

In some embodiments, the system may include the invasive patient interface. The invasive patient interface may be selected from a group including but not limited to: (i) an endotracheal tube; (ii) a laryngeal mask airway (LMA); and (iii) a tracheostomy tube.

In some embodiments, the system may include a humidifier configured to condition the gas to a pre-determined temperature and/or humidity before delivery to the patient. Alternatively/additionally, the system may include a filter configured to treat gases exiting from the patient before they are is released to atmosphere. Alternatively/additionally, the system may include a gas delivery conduit providing a flow of gas from the flow source to the invasive patient interface.

In some embodiments, the first respiratory support is provided during absence of spontaneous breathing. The system may be configured e.g. to provide the first respiratory support during a medical procedure requiring substantial stillness of the abdomen and/or thorax.

Viewed from another aspect, the present disclosure provides a system for providing respiratory support to a patient during a medical procedure, the system comprising: (a) a flow source configurable to provide a flow of gas for delivery to the patient via an invasive patient interface; and (b) a controller controlling the flow source; wherein the system is couplable with an invasive patient interface having a sealable member that is configurable to form a sealing engagement with the patient's airway; and wherein, responsive to the invasive patient interface being non-sealingly arranged within the patient's airway during absence of spontaneous breathing, the controller controls the flow source to provide a flow of gas for delivery to the patient. The flow of gas provided by the flow source may be e.g. substantially continuous.

The controller may be configurable to receive one or more inputs and to control the flow source according to the one or more received inputs. In some embodiments, the controller may be configured to receive one or more inputs indicative of one or more of: (a) arrangement of the invasive patient interface within the patient's airway; and (b) presence/absence of spontaneous breathing by the patient. Alternatively/additionally, the controller may be configured to receive one or more inputs comprising signals generated by one or more of, for example: (a) an exiting gases flow sensor; (b) a pressure sensor associated with an inflatable body comprising the sealable member; (c) an airway pressure sensor; and (d) a user input device.

In some embodiments, the controller may control the flow rate of gas provided by the flow source. The flow rate of gas provided by the flow source may be in a range of e.g. 0 LPM to about 120 LPM, preferably about 20 LPM to about 70 LPM, more preferably at least about 40 LPM. In some embodiments, the flow rate of gas provided by the flow source may be variable.

In some embodiments, the system may be operable to provide an alternative form of respiratory support, wherein responsive to the invasive patient interface being sealingly engaged with the patient's airway, the controller controls the flow source to provide a cyclic flow of respiratory gas for delivery to the patient. In some embodiments, the system may be operating to provide the alternative form of respiratory support prior to the system responding to the invasive patient interface becoming non-sealingly arranged within the patient's airway.

In some embodiments, the system may be operable to provide an alternative form of respiratory support, wherein responsive to the invasive patent interface being sealingly engaged with the patient's airway, the controller controls the flow source such that the respiratory support is dependent on or imitates an intended breathing cycle of the patient.

In some embodiments, the controller may determine the invasive patient interface to be non-sealingly arranged within the patient's airway upon detection of one or more of, for example: (a) gases exiting the patient's airway outside the invasive patient interface; (b) reduced inflation pressure of an inflatable body comprising the sealable member at or below a predetermined inflation pressure; (c) reduced pressure within the patient's airway; and (d) a user input confirming the non-sealing arrangement.

In some embodiments, the controller may determine spontaneous breathing by the patient to be absent upon detection of one or more of a parameter associated with gas flow to and/or from a patient's airway, movement of the patient's chest, and a user input confirming there is no spontaneous breathing by the patient. In some embodiments, the controller may generate an audible and/or visible alert to communicate detected absence of spontaneous breathing by the patient.

In some embodiments, the system may include the invasive patient interface. The invasive patient interface may be selected from a group including but not limited to: (i) an endotracheal tube; (ii) a laryngeal mask airway (LMA); and (iii) a tracheostomy tube.

In some embodiments, the respiratory support provided by the system maintains a period of safe apnoea while the invasive patient interface is non-sealingly arranged within the patient's airway.

Viewed from another aspect, the present disclosure provides system for providing respiratory support to a patient during a medical procedure, the system comprising: (a) a flow source configurable to provide a flow of gas for delivery to the patient via an invasive patient interface; and (b) a controller controlling the flow source; wherein the system is couplable with an invasive patient interface having a sealable member and configurable to form a sealing engagement with the patient's airway, or to form a non-sealing arrangement of the invasive patient interface within the patient's airway; wherein the non-sealing arrangement creates an exit flow path between an external wall of the invasive patient interface and the patient's airway for flow of gases exiting from the patient; and wherein the controller controls the flow source to achieve a target flow rate out of the exit flow path. In some embodiments, the target flow rate is at least about 0.5 LPM, preferably at least about 5 LPM or at least about 20 LPM.

In some embodiments, the exit flow path has a non-zero cross-sectional area at the sealable member.

In some embodiments, the exit flow path surrounds the invasive patient interface in the patient's airway and comprises a distance between the sealable member and the airway wall of at least about 1% of the airway radius, preferably at least about 2%, or at least about 3% or at least about 4% or at least about 5% of the airway radius.

In some embodiments, the controller receives inputs indicative of flow rate in the exit flow path and provides guidance for deflation of an inflatable body comprising the sealable member to achieve the target flow rate. The controller may control inflation of the inflatable body according to the received inputs indicative of flows in the exit flow path.

In some embodiments, the controller may receive inputs indicative of flow rate in the exit flow path and adjust control of the flow source to achieve the predetermined target flow rate. Alternatively/additionally, the controller may receive inputs indicative of pressure in the patient's airway and adjust control of the flow source to achieve a predetermined patient airway pressure. The predetermined airway pressure may include, for example, about 2 cmH₂O, or about 2 cmH₂O to about 20 cmH₂O, or about 2 cmH₂O to about 10 cmH₂O, or about 2 cmH₂O and about 5 cmH₂O, or about 5 cmH₂O to about 10 cmH₂O.

In some embodiments, the may controller receive inputs comprising signals generated by one or more of, for example: (a) an exiting gases flow sensor; (b) a pressure sensor associated with an inflatable body comprising the sealable member; (c) an airway pressure sensor; and (d) a user input device.

In some embodiments, the flow of gas provided by the flow source is substantially continuous. The flow rate of gas provided by the flow source may be in a range of about 0 LPM to about 150 LPM, preferably about 20 LPM to about 70 LPM, preferably at least about 40 LPM. The flow rate of gas provided by the flow source may be variable.

In some embodiments, the system may include the invasive patient interface which may be selected from a group including but not limited to: (a) an endotracheal tube; (b) a laryngeal mask airway (LMA); and (c) a tracheostomy tube.

Viewed from another aspect, the present disclosure provides a method for providing respiratory support to a patient during a medical procedure, the method comprising the steps of: (a) intubating the patient with an invasive patient interface having a sealable member operable to form a sealing engagement with the patient's airway; (b) while the patient is intubated, operating the sealable member to form a non-sealing arrangement of the invasive patient interface within with the patient's airway; and (c) providing a flow of respiratory gas to the patient via the invasive patient interface; wherein the non-sealing arrangement creates an exit flow path between an external wall of the invasive patient interface and the patient's airway for flow of gases exiting from the patient; and wherein the flow of respiratory gas is controlled to achieve a target flow rate out of the exit flow path.

In some embodiments, the target flow rate is at least about 0.5 LPM, preferably at least about 5 LPM or at least about 20 LPM.

In some embodiments, the exit flow path has a non-zero cross-sectional area at the sealable member. Alternatively/additionally, the exit flow path surrounds the invasive patient interface in the patient's airway and may include a distance between the sealable member and the airway wall of at least about 1% of the airway radius, preferably at least about 2%, or at least about 3% or at least about 4% or at least about 5% of the airway radius.

In some embodiments, the sealable member is an inflatable body, and the method includes deflating the inflatable body to achieve the target flow rate.

In some embodiments, the flow of respiratory gas is substantially continuous. The flow of respiratory gas may include a flow rate of about 0 LPM to about 120 LPM, preferably about 20 LPM to about 70 LPM or at least about 40 LPM. The flow of respiratory gas may have a flow rate that is variable.

In some embodiments, the respiratory support is provided during absence of spontaneous breathing. In some embodiments, the medical procedure requires substantial stillness of the abdomen and/or thorax.

It is to be understood that various features and aspects of the disclosed embodiments can be combined with or substituted for one another in order to form varying modes of the disclosed embodiments. Thus, it is intended that the scope of the present disclosure should not be limited by the particular aspects and feature combinations expressly disclosed, but should be determined as encompassing feature combinations not expressly disclosed but nevertheless understood upon fair reading of the specification to be suitable for combination in a manner similar to other aspects and embodiments disclosed elsewhere herein.

### BRIEF DESCRIPTION OF DRAWINGS

The invention will now be described in greater detail with reference to the accompanying drawings in which like features are represented by like numerals. It is to be understood that the embodiments shown are examples only and are not to be taken as limiting the scope of the invention as defined in the claims appended hereto.

FIG. 1 is a schematic illustration showing a system for providing respiratory support to a patient according to embodiments of the disclosure.

FIG. 2 is a flow chart showing steps in a method for providing respiratory support to a patient according to an embodiment of the disclosure which involves deflation of a usually inflated sealable member.

FIG. 3 illustrates a non-sealing arrangement of the invasive patient interface sealable member within the patient's airway.

FIG. 4 illustrates provision of a flow of respiratory gas via the invasive patient interface in the non-sealing arrangement of FIG. 3.

FIG. 5 illustrates an example of inflation means that may be used to operate an inflatable sealable member to form a substantially sealing engagement with the patient's airway, or a non-sealing arrangement within the airway.

FIG. 6 is a schematic illustration showing a system with a controller, for providing respiratory support to a patient according to embodiments of the disclosure.

FIG. 7 is a schematic illustration of a connector for use with an invasive patient interface according to embodiments of the disclosure.

FIG. 8 is a flow chart showing steps in a method for providing respiratory support to a patient involving switching between different forms of respiratory support.

FIG. 9 is a flow chart showing steps in a method for providing respiratory support to a patient involving switching between different forms of respiratory support provided by different flow sources.

FIG. 10 is a schematic illustration showing a controller configured to receive one or more inputs.

FIG. 11 is a flow chart showing in schematic form steps in a method for providing respiratory support to a patient during a medical procedure in which an objective is to achieve a target expiratory flow rate.

FIGS. 12a to 12h represent in illustrative form, steps in a medical procedure involving methods and systems of the present disclosure.

### DETAILED DESCRIPTION

Embodiments of the disclosure are discussed herein by reference to the drawings which are not to scale and are intended merely to assist with explanation of the invention.

For simplicity, the same reference numerals have been used throughout this specification for the systems 100 according to the inventive aspects as disclosed herein. Thus, the systems 100 as shown may encompass one or more features of the inventive aspects as described in relation to the various embodiments of the disclosure. Features of the systems 100 sharing the same reference numerals correspond to the same features as described in connection with the various embodiments of the disclosure.

Embodiments of the disclosure are generally directed to provision of respiratory support to a patient via an invasive patient interface, such as an endotracheal tube (ETT), laryngeal mask airway (LMA) or a tracheostomy tube. Typically, these invasive patient interfaces have a sealable member (typically an inflatable body or cuff) that is operable to seal with a patient's airway. Embodiments of the disclosure may be used during medical procedures in which the patient is anaesthetized and for which respiratory stillness is required. The respiratory support provides a flow of respiratory gas from a flow source to the patient's airway, via the invasive patient interface. In some embodiments, the respiratory support is provided at flow rates and/or velocities which are effective in clearing CO₂ from the patient and/or to provide pressure support to the patient. In various embodiments improved CO₂ clearance is achieved by modifying the condition of the invasive patient interface such that the sealable member (typically an inflatable body or cuff) is deflated opening an outflow path (which is normally closed for respiratory support) through which gases from the airway may exit.

An absence of movement of the diaphragm and/or thorax and/or abdomen may be termed ‘respiratory stillness’. However, the safe duration of a respiratory support during a period of respiratory stillness may be limited by CO₂ build up in the patient's blood which can be affected by CO₂ clearance in the lungs. In spontaneously breathing patients, the movement of the diaphragm inflates and deflates the lungs, where the deflation of the lungs creates expiratory movement of the gases in the patient's airway to clear CO₂. Mechanical ventilation can support ventilation in spontaneously breathing patients, for example the system could deliver a set pressure or volume of gases based on the patient's breathing pattern, to aid the clearance of CO₂. In non-spontaneously breathing patients supported by mechanical ventilation, breathing is mimicked and the control of pressure and/or volume of the mimicked expiratory phase allows for the clearance of CO₂ from the patient's airways, predominantly caused by elastic recoil from the previously inflated lungs. In the absence of an expiratory motion, whether via spontaneous breathing (with or without mechanical ventilator support) or via mechanical ventilation in a non-spontaneously breathing patient, CO₂ could build up in the patient because of inadequate gas mixing leading to insufficient CO₂ clearance. Furthermore, lack of breathing motion (both inspiratory and expiratory) whether via spontaneous breathing (with or without mechanical ventilator support) or via mechanical ventilation in a non-spontaneously breathing patient, could cause inadequate oxygenation. Beneficially, embodiments of the disclosure provide improved CO₂ clearance and/or improve oxygenation to provide a safer and prolonged period of respiratory stillness.

Beneficially, improved CO₂ clearance is achieved while the patient is in substantial respiratory stillness, and provision of respiratory support according to the embodiments disclosed herein causes little or no movement of the patient, in particular little or no movement to the patient's diaphragm and/or thorax and/or abdomen. Provision of respiratory support during a period of what may be termed “respiratory stillness”, while the patient is apnoeic and may be in the absence of mechanical ventilation, may be advantageous during medical procedures requiring stillness of the diaphragm, abdomen and/or thorax although utility of the disclosure is not limited to these circumstances. It is also worth noting that providing sufficient respiratory support during respiratory stillness can provide better access to some organs during specific medical procedures, for surgeons. These organs may be otherwise obscured if the lung is more inflated as is typically the case in conventional ventilation.

Advantageously, embodiments of the present disclosure permit provision of respiratory support in the absence of cycles of lung inflation and deflation (i.e. in the absence of ventilation involving movement of the diaphragm). Advantageously, embodiments of the present disclosure permit provision of respiratory support during a period of respiratory stillness. This provision of respiratory support occurs by providing oxygenation and CO₂ clearance and in some embodiments, pressure support. The CO₂ clearance benefits of embodiments disclosed herein are especially important for gas exchange during respiratory stillness as there is no breathing motion from lung inflation/deflation to clear CO₂.

In this specification, invasive patient interfaces include any device or instrument that is couplable with an airway of the patient, usually bypassing the patient's upper respiratory airway and having a sealable member or cuff that can be in a sealing or non-sealing state with the patient's airway or a portion thereof. In some embodiments, the invasive patient interface may be sealable with a portion of the patient's lower respiratory airway. In some embodiments, the invasive patient interface may be sealable with a lower portion of the patient's upper respiratory airway. Invasive patient interfaces include but are not limited to devices and instruments that penetrate via a patient's mouth, nose or skin to serve as an artificial airway, such as an endotracheal tube, tracheostomy tube or laryngeal mask airway to name a few. It will be appreciated that these are examples only, and that embodiments of the disclosure are not limited to use with endotracheal tubes or particular invasive patient interfaces described herein, and may employ other invasive patient interfaces with sealable members as would be understood by a person skilled in the art.

In this specification, the terms subject and patient may be used interchangeably. A subject or patient may refer to a human or an animal subject or patient.

In this specification, the terms “distal” and “proximal” are to be interpreted relative to the subject or patient. Distal refers to a feature being directed away from or further from the patient. Proximal refers to a feature being directed towards or close to the patient.

In this specification, the respiratory gas provided by a flow source could include, without limitation, oxygen, carbon dioxide, nitrogen, helium, and anaesthetic agents, to name a few, or mixtures of these or other breathable gases for respiration and/or ventilation. Where reference is made to a particular gas herein, it will be appreciated that it is by way of example only and the description can apply to any gas—not just that referenced.

Without limitation, some indicative values of flow rates for the respiratory gas provided by a flow source can be as follows.

In some configurations, the respiratory support includes provision of gases from a flow source to a patient at a flow rate of greater than 0 litres per minute (greater than 0 LPM or L/min). In some configurations, the respiratory support includes provision of gases from a flow source to a patient at a flow rate of about 5 or 10 LPM to about 150 LPM, or about 10 LPM to about 120 LPM, or about 15 LPM to about 95 LPM, or about 20 LPM to about 90 LPM, or about 20 LPM to about 70 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to various embodiments and configurations described herein, a flow rate of gases supplied or provided to a patient via a system or from a flow source, may comprise, but is not limited to, flows of at least about 5, 10, 15, 20, 30, 40, 50, 60, 70, 80, 90, 100, 110, 120, 130, 140, 150 LPM, or more, and useful ranges may be selected to be any of these values (for example, about 20 LPM to about 90 LPM, about 15 LPM to about 70 LPM, about 20 LPM to about 70 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

Flow rates at which gases are delivered from a flow source to a connector and/or delivered to a patient may be referred to as a therapeutic flow rate. The therapeutic flow rate can be time-varying (e.g. oscillating). That is, the therapeutic flow can have a time-varying (e.g. oscillating) flow rate component. This time-varying flow rate can aid respiratory support by providing improved oxygenation and/or CO₂ clearance and/or reduce the risk of atelectasis which may in turn lead to more evenly distributed patient pressure throughout the lung.

Flow rates for premature/infants/paediatrics (with body mass in the range of about 1 to about 30 kg) can be different. The flow rate can be set to about 0.4 L/min/kg to about 8 L/min/kg with a minimum of about 0.5 L/min and a maximum of about 70 L/min. For patients under 2 kg maximum flow rate is set to 8 L/min. Oscillating flow may be set to 0.05-2 L/min/kg with a preferred range of 0.1-1 L/min/kg and another preferred range of 0.2-0.8 L/min/kg

In some embodiments, the flow rates disclosed herein may generate a flushing effect in the patient's airway such that the anatomical dead space of the upper airways is flushed by the incoming gas flows. This can create a reservoir of fresh gas, and/or minimise the gas concentration of carbon dioxide, nitrogen, etc. which may help to improve CO₂ clearance and/or reduce the build up of CO₂. Incoming gas flows with higher than ambient amount of oxygen may expedite the time required to replace gases, e.g. carbon dioxide, nitrogen, etc in the patient's lungs and/or increase the patient's blood oxygen saturation levels.

Gases delivered may comprise a percentage of oxygen. In some configurations, the percentage of oxygen in the gases delivered may be about 15% to about 100%, 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Respiratory Support with Cuff Deflation

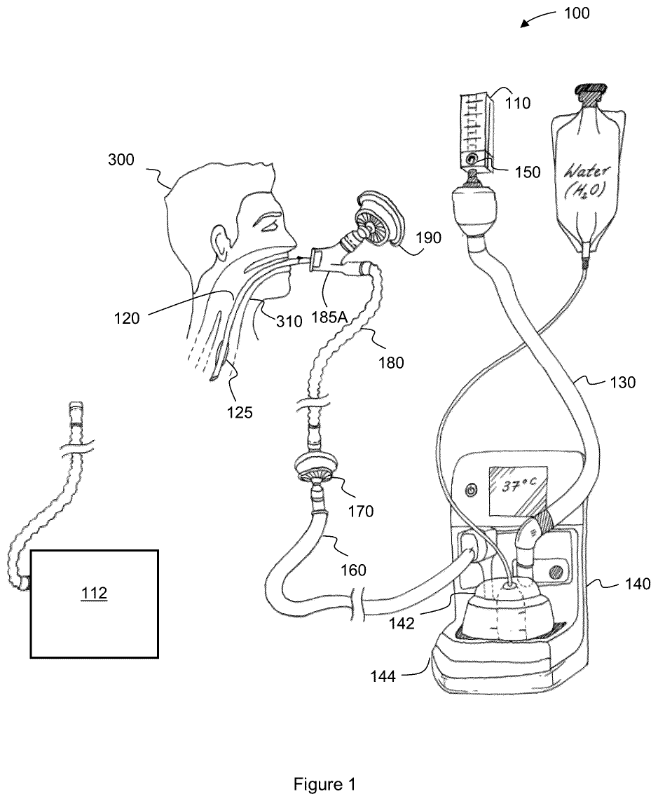

FIG. 1 is a schematic illustration showing a system 100 for providing respiratory support to a patient 300 according to embodiments of the disclosure. System 100 includes a flow source 110, which in the embodiment shown includes a flow meter receiving a flow of gas from an O₂ source and/or air source, and provides a flow of respiratory gas to an invasive patient interface 120 and to the patient 300.

The gas flow is delivered from the flow source 110 to a conduit 130 connectable between the flow source 110 and a humidifier 140. In some embodiments, the flow source 110 and humidifier 140 exist as separate components in the respiratory system and in other embodiments, the flow source 110 and 140 humidifier may be provided in a single unitary component. In the embodiment of system 100 shown, the humidifier 140 includes a humidification chamber 142 and a humidification base unit 144. The conduit 130 may be referred to as a dry line for delivering a flow of dry gases from the flow source 110 to the humidifier 140. The conduit 130 may be coupled to an inlet of the humidification chamber 142 of the humidifier 140 as shown. In alternative embodiments, the humidifier 140 may be a single component (not shown) and exclude the separate humidification chamber 142 and base unit 144. The humidifier 140 may be configured to condition the gas provided by the flow source 110 to a selected temperature and/or humidity, for example, within the humidification chamber 142 as shown. The temperature and/or humidity selected may be dependent on the respiratory support being provided and may be selected by the user or operator.

The conditioned gas flow travels from humidifier 140 (or more specifically the humidification chamber 142) as shown through inspiratory conduit 160 which is connectable between humidifier 140 and invasive patient interface 120. Coupling may be by, for example a wye piece connector 185A although other connectors are also contemplated. In other embodiments, system 100 may omit any humidifier component and the flow source 110 may be directly couplable, or couplable through an interface conduit 180, with invasive patient interface 120.

The flow source 110 may include a compressed gas source, a device that modifies flow from a compressed gas source and/or a flow generator which generates a gas flow. Ideally, the flow source 110 provides flow at therapeutic flow rates including between about 5 or 10 LPM and about 150 LPM. More preferably, the therapeutic flow rates are between about 20 LPM and about 70 LPM. The therapeutic flow rates may be between about 40 LPM and about 70 LPM. The range of flows provided from flow source 110 to achieve sufficient patient oxygenation and CO₂ clearance as well as maintain a suitable patient pressure and a desirable expiratory resistance may be between about 20 LPM and about 70 LPM. However, this range is dependent on the patient 300 being supported by the system 100, for example infants and children may not tolerate as high flow rates, and will require lower rates, as defined previously. Preferably for infants and children, the therapeutic flow rate may be in a range of about 0.5 L LPM to about 25 LPM.

The gas provided to the patient 300 may be flow rate controlled. In some embodiments, the flow source 110 is configured to provide a continuous flow of gas at the selected flow rate. The continuous flow may be a unidirectional or positive net flow towards the patient. Furthermore, the therapeutic flow rate for the flow source 110 may be a fixed flow rate. The fixed flow rate may be independent of the respiratory cycle of the patient 300.

In other embodiments, the flow source 110 may be configured to provide a flow of gas at a therapeutic flow rate that is time-varying (e.g. oscillating) and a controller controls a flow modulator to provide the therapeutic time-varying gas flow with an oscillating flow rate of: about 375 litres/min to about 0 litres/min, or preferably of about 240 litres/min to about 7.5 litres/min, or more preferably of about 120 litres/min to about 15 litres/min, and/or the oscillating flow rate has one or more frequencies of about 0.1 Hz to about 200 Hz, and preferably about 0.1 Hz to about 6 Hz, and more preferably about 0.5 Hz to about 4 Hz, and more preferably 0.6 Hz to 3 Hz. The gas flow modulator may comprise be the flow source 110 (where that could be a flow generator, O₂ source, ambient air or the like as previously discussed) and/or a valve or other device to modulate or otherwise vary parameters (e.g. flow rate, gas proportion) of a gas flow.

The oscillating flow rate may comprise a therapeutic flow rate component, wherein the therapeutic flow rate is about 375 litres/min to about 0 litres/min, or about 150 litres/min to about 0 litres/min, or is preferably about 120 litres/min to about litres/min, or is more preferably about 90 litres/min to about 30 litres/min. The oscillating flow rate may comprise a therapeutic gas flow component, wherein the constant (e.g. bias/base) flow rate component of the therapeutic gas flow is about 0.5 litres/min to about 70 litres/min. In some configurations, the therapeutic flow rate component is time-varying.

The oscillating flow rate may comprise a therapeutic flow rate component, wherein the therapeutic flow rate is about 0.2 litres/min per patient kilogram to about 2.5 litres/min per patient kilogram; and preferably is about 0.25 litres/min per patient kilogram to about 1.75 litres/min per patient kilogram; and more preferably is about 0.3 litres/min per patient kilogram to about 1.25 litres/min or about 1.5 litres/min per patient kilogram; and more preferably is about 0.4 litres/min per patient kilogram to about 0.8 litres/min per patient kilogram.

The one or more components of the time-varying (e.g. oscillating) gas flow may have one or more frequencies of about 0.3 Hz to about 4 Hz.

The oscillating flow rate may comprise at least one time-varying flow rate component, wherein each oscillating flow rate is about 0.05 litres/min per patient kilogram to 2 litres/min per patient kilogram; and preferably is about 0.05 litres/min per patient kilogram to about 0.5 litres/min per patient kilogram; and preferably about 0.12 litres/min per patient kilogram to about 0.4 litres/min per patient kilogram; and more preferably about 0.12 litres/min per patient kilogram to about 0.35 litres/min per patient kilogram. Alternatively, the oscillating flow rate may comprise at least one time-varying flow rate component, wherein each oscillating flow rate is in the range of 0.05 litres/min per patient kilogram to 2 litres/min per patient kilogram; and preferably in the range of 0.1 litres/min per patient kilogram to 1 litres/min per patient kilogram; and more preferably in the range of 0.2 litres/min per patient kilogram to 0.8 litres/min per patient kilogram.

The above are just examples, and other types of time-varying flow rates could be provided with the controller controlling a gas flow modulator to provide the time varying gas flow with the time-varying flow rate. The controller may have knowledge of the time-varying flow rate, and/or may measure the time-varying flow rate provided, e.g. by one or more flow sensors downstream of the flow modulator.

System 100 may include an optional filter 170. The filter 170 may be positioned between the inspiratory conduit 160 and the patient interface conduit 180. Gases flowing through the inspiratory conduit 160 are passed to the patient by way of the optional filter 170 and the invasive patient interface 120 which may be coupled with patient interface conduit 180 either directly or indirectly e.g. using an adapter. In some embodiments, the inspiratory conduit 160 and patient interface conduit 180 may be a single conduit with or without a filter 170.

System 100 may include another optional filter 190 which may be couplable to the outlet port 230 of the invasive patient interface or connector 185A for filtering the gases exiting from the airway through the interface, as shown in FIG. 1. A filter 190 may be used e.g. if the patient 300 is provided with gases containing nebulized drugs that can be harmful to surrounding personnel or to the environment. The filter 190 preferably captures contaminants, aerosols, pathogens, etc. in the exit gas flow directed through it.

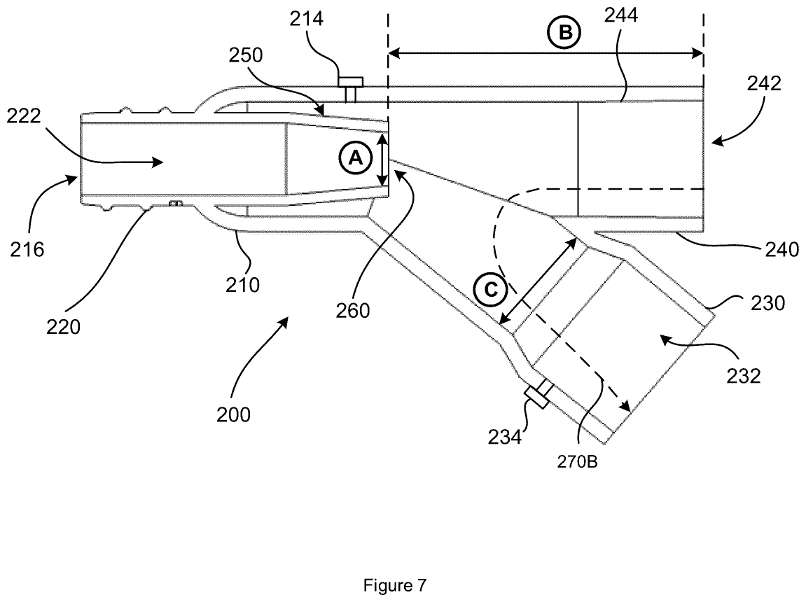

In some embodiments, the filters 170 and 190 may be non-removable and/or integral with conduit 160 and connector 185A, respectively. Alternatively, the filters 170 and 190 may be releasably couplable with the conduit 160 and connector 185A, respectively. In some embodiments, wye piece connector 185A may be replaced with or used in conjunction with a connector having one or more constrictions configured to generate an intended effect, for example a predetermined airway pressure. An example of one such connector is described in relation to FIG. 7 below.

The inspiratory conduit 160 and patient interface conduit 180 may be one or more of corrugated, flexible, bendable, resistant to kink and/or heated (e.g., the conduits 160, 180 may include a heating element). Additionally/alternatively, the conduit 180 or a portion thereof may be formed of a breathable material, such as described in U.S. Pat. No. 7,493,902 which is incorporated herein by reference.

In the context of the present disclosure, expired gases refers to gases that have been expired from the lungs. Gases exiting the patient's airway may or may not have been subjected to inhalation/exhalation motion as part of a spontaneous or non-spontaneous (i.e. artificially ventilated) respiratory cycle. Thus, gases exiting the patient's airway may include inspiratory gases which have not undergone alveolar gas exchange, old gases including those that have and have not been exchanged, as well as exchanged gases containing CO₂ as a result of alveolar gas exchange, irrespective of whether there has been inhalation/exhalation as part of a respiratory cycle.

In the context of the present disclosure, inspiratory conduit 160 permits the provision of gases to the patient via invasive patient interface 120 (and optionally filter 170 and interface conduit 180) for the provision of respiratory support. The respiratory support may include inhalation and/or exhalation as part of a spontaneous or non-spontaneous (i.e. artificially ventilated) respiratory cycle although that need not be the case. In many embodiments disclosed herein, respiratory gases conveyed by the inspiratory conduit 160 are provided as part of a respiratory support that is independent of cycles of spontaneous or non-spontaneous (i.e. artificially ventilated) inspiratory or expiratory breathing motion. As disclosed elsewhere herein, the respiratory support may comprise a flow of gases at a constant (albeit variable) flow rate, and/or a flow of gasses that is time-varying or oscillatory.

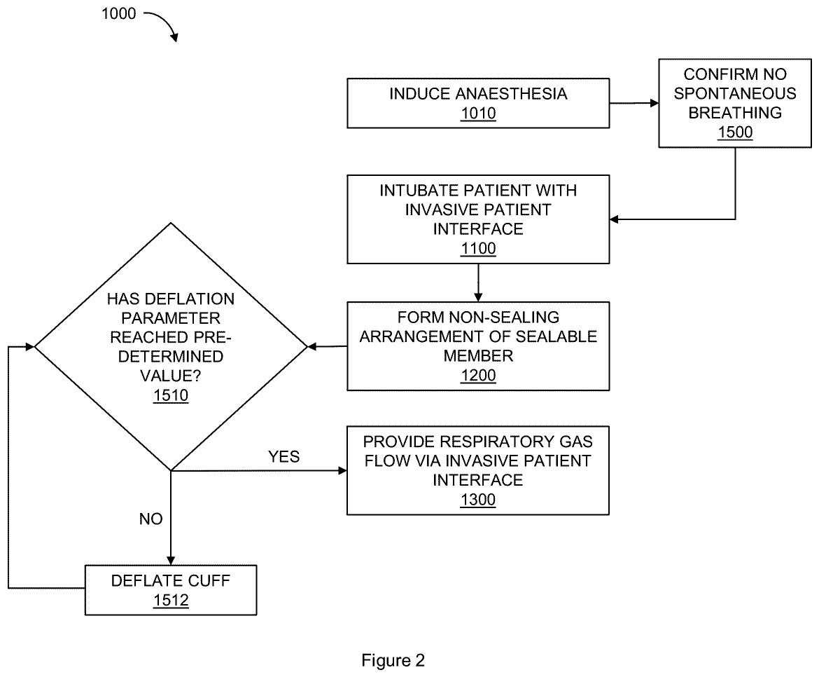

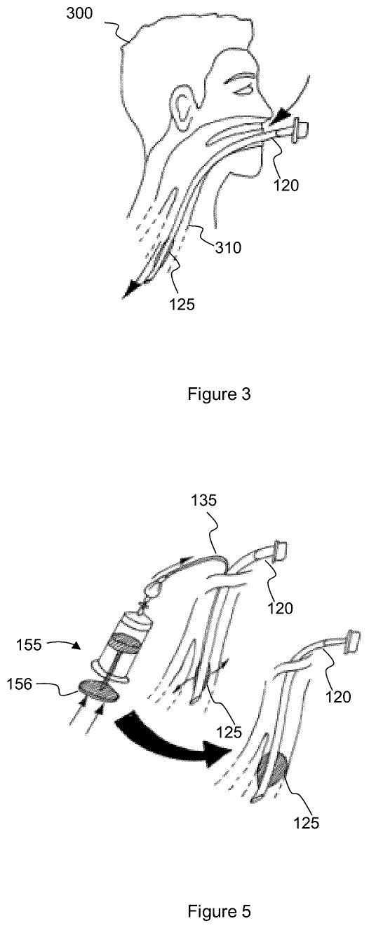

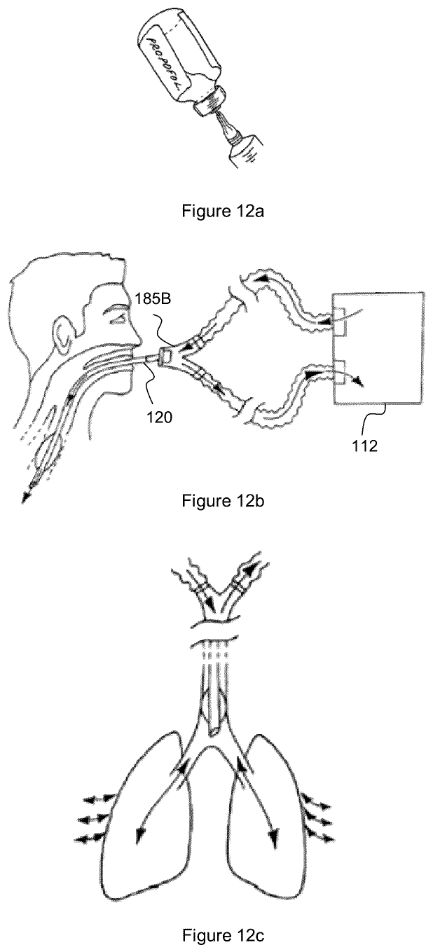

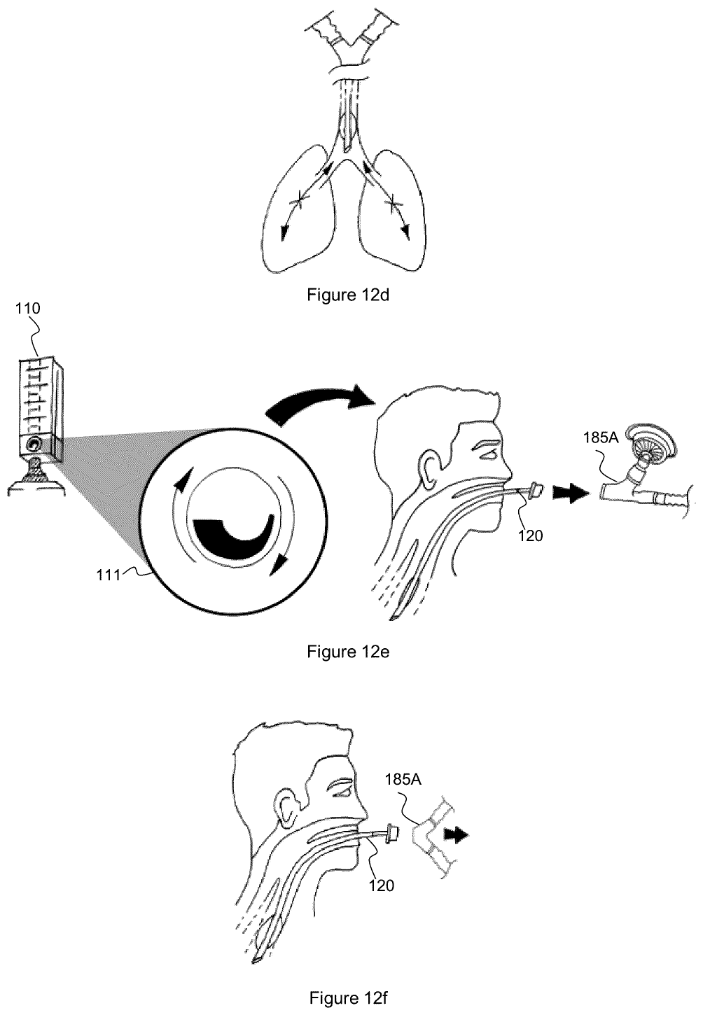

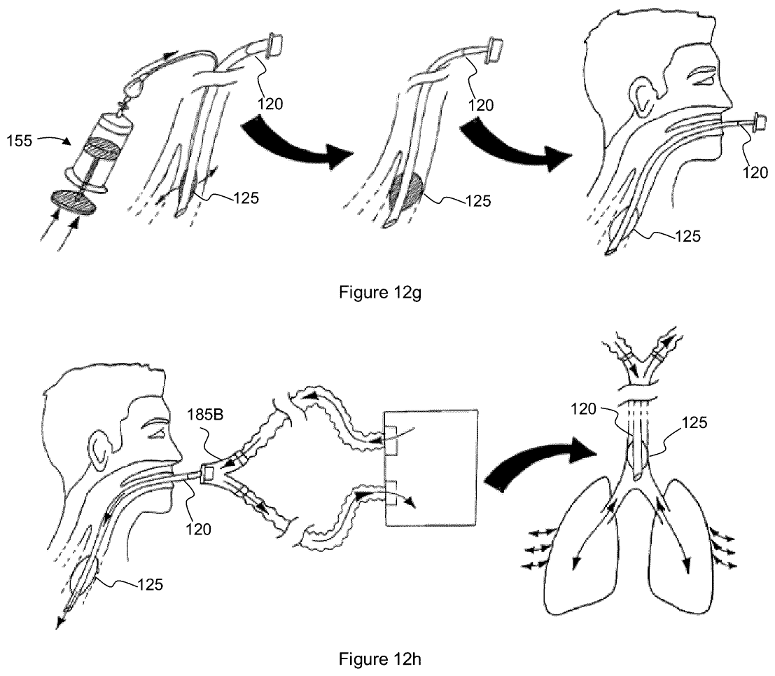

Referring to FIG. 2, a flow chart shows in schematic form steps in a method 1000 for providing respiratory support to a patient according to an embodiment of the disclosure which involves deflation of a cuff or inflatable member of an invasive patient interface which is typically inflated for provision of ventilatory respiratory support. In a step 1100, patient 300 is intubated (FIG. 3) with an invasive patient interface 120 having a sealable member 125 that is operable to form a sealing engagement with the patient's airway (see FIG. 12g below). Typically, the sealable member 125 is an inflatable body such as a balloon or cuff attached to an outside wall of the invasive patient interface 120 toward its proximal end. The terms sealable member, inflatable body, balloon and cuff may be used interchangeably herein and are designated the common reference numeral 125 in the drawings.

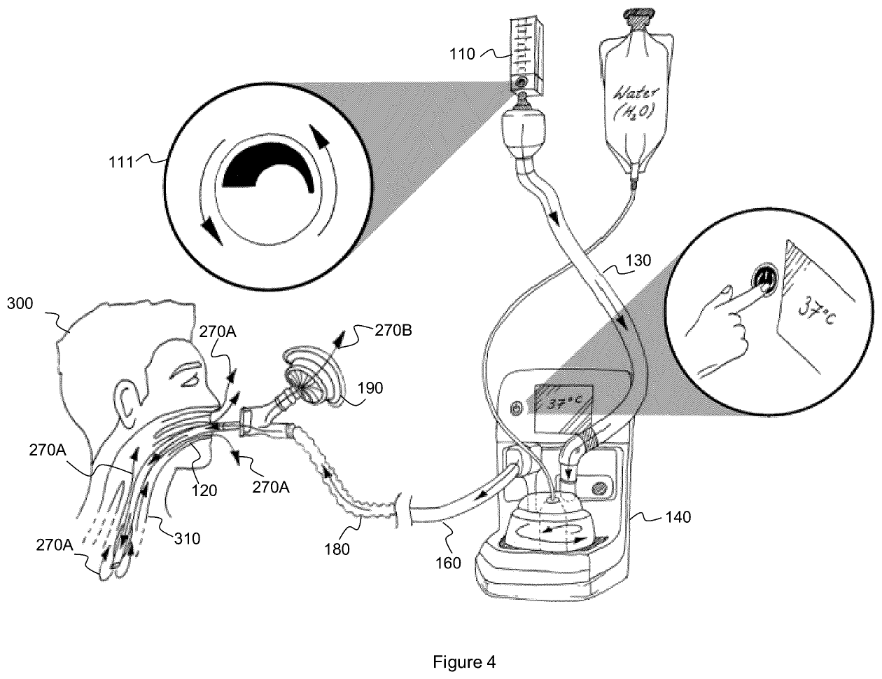

The cuff 125 is operable to be inflated to substantially seal against the walls of the patient's airway 310 to secure its location e.g. for use in manual or mechanical ventilation, or to be deflated e.g. prior to removal from the airway 310. When inflated in the airway, cuff 125 also provides a sealed system for the provision of pressure/volume controlled mechanical ventilation, and reduces risks associated with gastric aspiration. According to method 1000, in a step 1200 and while the patient is intubated, cuff 125 is operated (which may include deflation from an inflated state) to form a non-sealing arrangement of the invasive patient interface 120 within with the patient's airway 310. The arrangement of the sealable member 125 within the patient's airway 310 in the non-sealing arrangement is illustrated in FIG. 3. In a step 1300 a flow of respiratory gas is provided to the patient via the invasive patient interface 120 (FIG. 4). In some embodiments the flow is provided to the invasive patient interface 120 using a connector having one or more constrictions configured to generate an intended effect, for example a patient airway pressure. One example of such a connector in which the one or more constrictions comprise a nozzle is described in relation to FIG. 7. Other examples of suitable connectors are disclosed in commonly owned U.S. Provisional Patent Application 63/079,651 filed 17 Sep. 2020, which is hereby incorporated herein by reference.

FIG. 5 illustrates an example of inflation means 155 that may be used to operate the cuff 125 to form a substantially sealing engagement with the patient's airway 310, or a non-sealing arrangement within the airway as required for the form of respiratory support provided according to the method 1000. In this example, inflation means 155 includes a syringe which is in fluid communication with cuff 125 by a tube 135. Plunger 156 is advanced within the syringe 155 to force a fluid, e.g. air into tube 135 and cuff 125 to achieve inflation, and retraction of plunger 156 withdraws air from the cuff to achieve deflation.

While a syringe pump is exemplified in the figures, it is to be understood that a variety of pump types including a manual bulb pump and electronically controlled pump may be suitable to achieve inflation and/or deflation of the cuff 125. Furthermore, the cuff may be inflated using a gaseous or liquid fluid. Gaseous fluids (such as air or air/oxygen blends) may be vented to (or drawn from) atmosphere to deflate (or inflate) the cuff or they may be part of a closed system, as in the case of a syringe pump. It is to be understood, however, that the sealable member need not be inflatable/deflatable, and may use characteristics or behaviours of certain materials (e.g. foams or the like) to form the sealing and non-sealing states within the airway.

For a sealable member in the form of an inflatable cuff, inflation is required to achieve a substantially sealing engagement of cuff with the patient's airway and this typically involves inflating the cuff to a pressure of about 20 cmH₂O to about 30 cmH₂O. Deflation is required (after inflation) to achieve the non-sealing arrangement of the invasive patient interface 120 within the patient's airway as required for provision of respiratory support according to method 1000. Deflation to a cuff pressure less than the sealing pressure (e.g. less than about 20 cmH₂O) achieves the non-sealing arrangement although a lower predetermined inflation pressure may be preferred to increase CO₂ clearance. In some embodiments, a preferred predetermined inflation pressure is about minus 10 cmH₂O relative to atmosphere.

Deflation of cuff 125 to form the non-sealing arrangement forms a first exit flow path 270A between an external wall of the invasive patient interface 120 and the patient's airway 310 for outflow of gases from the patient's airway. This is best shown in FIG. 4. It is to be understood that during provision of respiratory support according to method 1000, the patient may be neither spontaneously breathing nor ventilated with artificial breaths intended to mimic or coincide with inspiratory and expiratory phases of spontaneous breathing. Therefore, gases flowing out from the patient's lungs and airway may not necessarily comprise “expired” gases in the sense that they are produced during the physical act of expiration. Rather, the first exit flow path 270A provides for exit of gases from the airway which may include inspiratory gases which have not undergone alveolar gas exchange, old gases including those that have and have not been exchanged, as well as exchanged gases containing CO₂ as a result of alveolar gas exchange, irrespective of whether there has been inhalation/exhalation motion as part of a respiratory cycle.

Providing respiratory support according to method 1000 enables gases in the airway to be removed by an exit flow path 270A that is around the invasive patient interface 120 and may exit via the mouth and/or nares. This increases CO₂ clearance from the patient during periods of respiratory support that require respiratory stillness, compared with use of an invasive patient interface with the cuff 125 inflated such that the sole exit pathway for gases in the airway is via the invasive patient interface 120 e.g. for a given therapeutic flow rate. It is to be understood, however that exit of gases through the invasive patient interface 120 is not precluded according to method 1000 and the invasive patient interface may provide a second exit flow path 270B for outflow of gases from the patient's airway through the invasive patient interface 120 in addition to providing a flow of respiratory gas to the patient. While FIG. 4 shows gases in exit flow path 270B exiting via filter 190, in some arrangements the filter may be omitted and/or an expiratory conduit may direct exiting gases to an exhaust port or elsewhere for treatment or removal from the surrounding environment. Exiting gases in the second exit flow path 270B may flow at a second predetermined flow rate which may be lower than the first predetermined flow rate in the first exit flow path 270A.

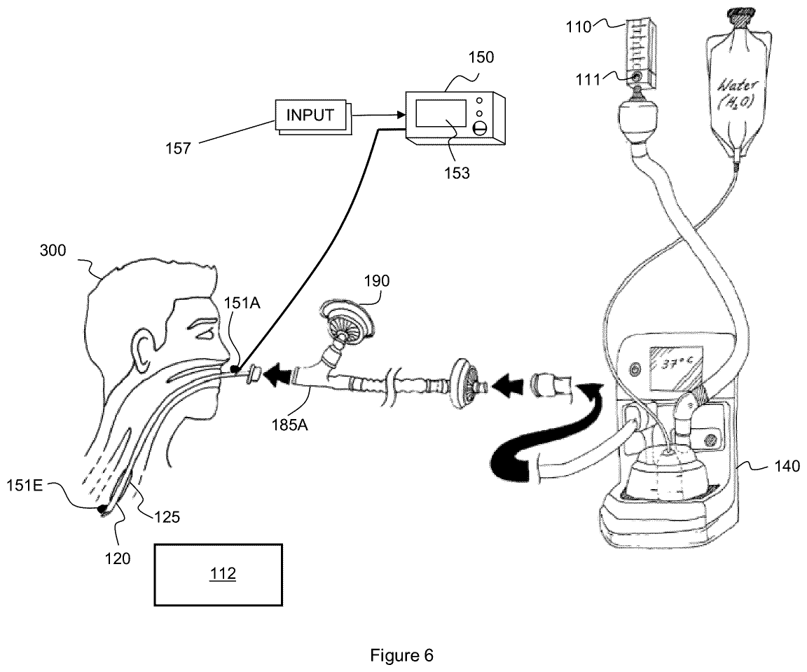

Preferably, gases in the first exit flow path 270A have a first non-zero predetermined flow rate which may be associated with increased CO₂ clearance and in some embodiments, may be at least about 0.5 LPM, preferably at least about 5 LPM and in some embodiments at least about 20 LPM. The predetermined flow rate in the first exit flow path 270A may be influenced, at least in part, by the rate of flow of respiratory gas that is provided via the invasive patient interface 120 in step 1300. In some arrangements, the predetermined flow rate in the first exit flow path 270A may be controlled by an automated control device such as controller 150 (FIG. 6). This may be achieved by the controller adjusting one or more variables of the flow provided from the flow source. In one embodiment, the controller adjusts rate of flow provided from the flow source to achieve the desired predetermined flow rate in the first exit flow path 270A. In embodiments where flow source 110 includes a blower, increasing or decreasing the flow rate provided from the flow source may be achieved by the controller 150 e.g. increasing or decreasing the motor speed of the blower. The flow rate of the gases in exit flow path 270A may be obtained by a sensor 151A and provided to the controller 150.

The predetermined flow rate may be programmed into controller 150 according to a protocol that is pre-programmed and stored in memory (locally or remotely) that is readable by the controller and preferably selectable by a user operating a user input device 152. Alternatively, a user may enter the predetermined flow rate or select a therapeutic flow rate from a list of available pre-programmed flow rates using user input device 152. Typically, the therapeutic flow rate is in a range of 0 LPM to about 150 LPM, preferably about 20 LPM to about 70 LPM and in some embodiments, preferably about 40 LPM. In some embodiments the therapeutic flow rate may be a variable flow rate, or it may be varied upon input from a user during provision of the respiratory support. It may be preferred that the flow of respiratory gas provided to the patient interface 120 during performance of method 1000 is substantially continuous in that it does not depend on an spontaneous or non-spontaneous (i.e. artificially ventilated) inspiratory or expiratory breathing cycles of breathing for the patient. In some embodiments, the therapeutic flow rate may be time-varying (e.g. oscillating) or it may have a time-varying (e.g. oscillating) flow rate component.

In some embodiments, the respiratory gas is provided out of the invasive patient interface 120 and into the patient's airway 310 at a predetermined velocity. In some embodiments, the predetermined velocity comprises a velocity of gases exiting the invasive patient interface 120. In some embodiments, predetermined velocity comprises a velocity of gases provided by the invasive patient interface 120 proximal to or at the patient's carina, one or more bronchi and/or one or more bronchioles.

The predetermined velocity may be programmed into controller 150 according to a protocol that is pre-programmed and stored in memory (locally or remotely) that is readable by the controller and preferably selectable by a user operating a user input device 152. Alternatively, a user may enter the predetermined velocity or select it from a list of available pre-programmed velocities using user input device 152. The controller 150 may control one or more components to provide the gas out of the invasive patient interface 120 at the predetermined velocity, for example, controlling the flow rate of gases provided by the flow source 110. In some embodiments, a user may provide inputs representing parameters of the respiratory support system to the controller 150, and the controller may be pre-programmed with a look up table or algorithm stored in memory (locally or remotely) that is readable by the controller and determines automatically the flow rate required to be delivered by the flow source 110 in order to achieve the predetermined velocity. Parameters that may be provided to the controller by a user may include, for example, type and/or size/dimensions (e.g. length and diameter) of the invasive patient interface. In some embodiments, controller 150 is configured to receive a signal from a gas flow velocity sensor 151E which is used by the controller to control the flow source 110 to achieve the predetermined velocity. In some embodiments, gas flow velocity sensor 151E is located at the proximal (i.e. outlet) end of the invasive patient interface 120 through which respiratory gas is provided to the patient's airway in order to obtain a measurement of velocity of gases exiting the invasive patient interface 120.

It is to be understood that a clinician may determine optimal depth of insertion of invasive patient interface 120 for CO₂ clearance, and position the interface accordingly. Ideally, this is with the proximal end of the invasive patient interface at a target location which is near the carina or deeper in one or more bronchi and/or bronchioles or with the flow of gas directed into one or more bronchi and/or bronchioles. The clinician may reposition the invasive patient interface during delivery of respiratory support, particularly while the sealable member is in the non-sealing arrangement since in this arrangement, the invasive patient interface may be prone to movement within the airway, including retracting orally. Repositioning the invasive patient interface and in particular, advancing it deeper in the airway during cuff deflation and/or during provision of respiratory support with the cuff deflated may be beneficial in embodiments disclosed herein, including those embodiments that involve switching between different forms of respiratory support corresponding to the different configurations (non-sealing arrangement vs sealed engagement) of the invasive patient interface within the patient's airway.

Typically, the predetermined velocity is in a range of up to about 60 m/s, or up to about 30 m/s, preferably at least about 5 m/s, and in some embodiments preferably at least about 10 m/s. These predetermined velocities may be achieved at a therapeutic flow rate of about 20 L/min to about 70 L/min provided by the flow source 110. In some embodiments the predetermined velocity may be a variable velocity, or it may be varied upon input to the controller 150 from a user during provision of the respiratory support.

Without being limited by theory, providing respiratory support near or at the target location at, at least, a predetermined velocity, enhances gas mixing where gas exits the invasive patient interface. Movement of the gas out of invasive patient interface 120 at the predetermined velocity (or faster) creates gas separation at the exit of the invasive patient interface. This generates turbulence that induces gas mixing which in turn results in fresh gas delivered from invasive patient interface mixing with old and expired gas (being gas that has been expired from the lungs) in the patient's airway. The turbulence generated can be influenced by geometric features at the proximal end of the invasive patient interface. For example, an abrupt proximal end may provide a “sharp” edge that causes flow separation as the gas exits the interface thus generating turbulence. Higher velocity flows may generate more turbulence and more gas mixing. First exit flow path 270A provides for removal of those mixed gases around the invasive patient interface. Because exiting gases will seek the path of least resistance, opening first exit flow path 270A by deflating the cuff 125 provides for improved CO₂ clearance.

Put another way, operating the sealable member to move from a sealing engagement with the patient's airway to a non-sealing arrangement within the patient's airway (e.g. by deflating a balloon or cuff or the like), provides a new outflow path for mixed gases in the patient's lungs as well as new respiratory gases being delivered by the invasive respiratory interface. Converting to the non-sealing arrangement has the effect of reducing the resistance to flow (RTF) of gases flowing through the invasive patient interface to the patient so that more gas flows in this flow path. Thus, transitioning from the sealing engagement to the non-sealing arrangement (e.g. by deflating a balloon or cuff) reduces the RTF, which increases flow rate through the invasive patient interface which in turn increases the velocity of gases exiting the invasive patient interface also increasing CO₂ clearance. When gases exit the invasive patient interface at a target location deep in the patient's airway such as at or near the carina or deeper in one or more bronchi and/or bronchioles, the CO₂ clearance can be more effective.

It is to be understood that several factors contribute to enhanced CO₂ clearance in various embodiments and aspects of the disclosure and these factors can interrelate. One factor is the velocity of gases at a target location within the patient's airway. Increasing the velocity of gases at a target location within the patient's airway increases CO₂ clearance. Within a patient's airway includes within the invasive patient interface, at an end of the invasive patient interface, and outside of the invasive patient interface but within the patient's airway (for example between the end of the invasive patient interface and the patient's carina). In some configurations, the target location is at or close to the carina. The velocity of gases at the target location may be based on one or more exit velocities (e.g. average velocity) of gases exiting the invasive patient interface for delivery to the target location, and the distance of the proximal end of the invasive patient interface from the target location (e.g. the carina). The exit velocity of gases exiting the invasive patient interface may be influenced by factors such as flow rates at which gases are provided to the invasive patient interface, the resistance to flow of the flow path in which gases flow to the patient (e.g. cross sectional area of the flow path and/or the geometry of the internal walls of the lumen defining the flow path and/or the length of the flow path, and/or the presence or absence of constrictions in the flow path), the pressure gradient across which the gases flow, the flow rate of gases exiting the patient, the velocity of gases exiting the patient and the resistance to flow of gases exiting the patient. In some embodiments, the velocity of gases exiting the patient may be influenced by the exit velocity of gases exiting the invasive patient interface. In some scenarios, a desirable exit velocity of gases exiting the invasive patient interface is achieved about 5 mm from the carina since there is a risk of damage if gases are delivered too close to or at the carina. In most medical procedures however, a clinician locates the proximal end of the invasive patient interface (e.g. ETT) about 4 cm from the carina which reduces the likelihood of gases being delivered too close to or at the carina. Suitable exit velocities may include e.g. a velocity in a range of above about 0 m/s to about 25 m/s at a selected flow rate of about 5 L/min to about 70 L/min of the flow of gas provided to the invasive patient interface, such as a velocity in the range of about 5 m/s to about 20 m/s. In some embodiments, the exit velocity is a velocity in a range of about 10 m/s to about 15 m/s.

Another factor that contributes to enhanced CO₂ clearance in various embodiments and aspects of the disclosure is the flow rate of gases exiting the patient. Increasing this flow rate of gases exiting the patient increases CO₂ clearance. The flow rate at which gases exit the patient is related to the flow rate at which gases are provided to the patient through the invasive patient interface. The resistance to flow within the flow path may affect the flow rate delivered to the patient. For a given driving pressure of the flow source, altering the resistance to flow in flow path may alter the flow rate being delivered to the patient and hence alter the flow rate exiting the patient, either through or around the invasive patient interface, depending on the respiratory support being provided. Resistance to flow can be influenced by parameters of the flow path in which gases flow to the patient (e.g. cross sectional area of the flow path and/or the geometry of the internal walls of the lumen defining the flow path and/or the length of the flow path). As indicated above, a flow rate of gases provided by the flow source may be in a range of 0 to about 150 LPM, preferably about 20 LPM to about 70 LPM. In some embodiments, the flow rate of gas provided by the flow source is at least about 40 LPM and in many applications is about 70 LPM. Part of or all of the flow rate of gases provided by the flow source may be provided to the patient (via the lumen). In some configurations, the flow rate of gases exiting the patient is inversely related to the resistance to flow of the exit flow path from the patient to atmosphere, for example, the higher the resistance to flow, the lower the flow rate. This resistance to flow can be influenced by parameters of the exit flow path from the patient to atmosphere (e.g. cross sectional area of the flow path and/or the geometry of the internal walls of the structure defining the flow path and/or the length of the flow path). The resistance to flow of the exit flow path from the patient to atmosphere is related to pressures delivered to the patient, for example the greater the resistance to flow in the exit flow path, the greater the delivered pressures.

Another factor that contributes to enhanced CO₂ clearance in various embodiments and aspects of the disclosure is the pressure differential between inside the patient's airway and atmosphere. A pressure differential can affect flow rate of gases exiting the patient's airway. The pressure differential between inside the patient's airway and atmosphere may be influenced by factors such as flow rates at which gases are provided to the invasive patient interface, the resistance to flow of the flow path in which gases flow to the patient (e.g. cross sectional area of the flow path and/or the geometry of the internal walls of the lumen defining the flow path and/or the length of the flow path, and/or the presence or absence of constrictions in the flow path), the flow rate of gases exiting the patient, the velocity of gases exiting the patient and the resistance to flow of gases exiting the patient. The pressure differential may be determined by measuring the difference between pressure outside the proximal end of the invasive patient interface and pressure within the invasive patient interface, or the difference between pressure outside the proximal end of the invasive patient interface and atmosphere (as may be measured outside the gases flow path). Pressure measurements may be obtained using pressure sensors at these locations.

Use of the invasive patient interface to provide a flow of gas at a target location deeper in the patient's airway e.g. close to the patient's carina or deeper, in the patient's bifurcated airway, can increase CO₂ clearance. This, in conjunction with the exit flow path which is formed around the invasive patient interface when the sealable member is in the non-sealing arrangement, can be effective in achieving improved CO₂ clearance. Furthermore, in some embodiments, providing the flow of gas at, at least, the predetermined velocity and/or a predetermined airway pressure, can contribute to further improved CO₂ clearance.

In some embodiments, the respiratory support provided via the invasive patient interface 120 according to the method 1000 achieves a predetermined airway pressure. The predetermined airway pressure may also be termed a predetermined patient pressure. This may be beneficial for achieving and/or maintaining desired airway patency for a given range of flow rates, assisting with lung recruitment, preventing and/or mitigating atelectasis and/or improving oxygenation. The predetermined airway pressure may be greater than 0 cmH₂O, for example in a range of about 2 cmH₂O to about 20 cmH₂O, preferably about 2 cmH₂O to about 10 cmH₂O, and more preferably about 2 cmH₂O to about 5 cmH₂O. Together with provision of flow through the invasive patient interface 120 and exiting of gases through first exit flow path 270A, the predetermined patient pressure may help improve CO₂ clearance. In some embodiments, the predetermined airway pressure is achieved by use of a connector 200 having one or more constrictions, one example of which is illustrated in FIG. 7.

In some embodiments it may be desirable to determine that the cuff 125 has been deflated in the non-sealing engagement to a degree that provides sufficient gases flow in a first exit flow path 270A formed between the patient's airway 310 and the invasive patient interface 120 and cuff 125. Thus, it may be desirable to monitor a parameter indicative of a degree of inflation of cuff 125 in step 1510 to ascertain if the non-sealing arrangement has been established. If the monitored parameter indicates that the desired non-sealing arrangement has not yet been reached, cuff deflation continues at 1512. If the parameter indicates that the desired non-sealing arrangement has been reached, deflation of cuff 125 ceases and a flow of respiratory gas comprising a flow-controlled form of respiratory support is provided via the invasive patient interface 120 at step 1300. In circumstances where the monitored parameter indicates that there has been over-deflation of cuff 125, the controller 150 may progressively re-inflate the cuff 125 while continuing to monitor the parameter to determine if a required level of re-inflation has been reached. Alternatively/additionally, the controller 150 may provide an audible or visible alert where there has been over-deflation and a clinician or other operator may manually reinflate the cuff 125 to the required level of inflation.

A clinician or other attendant may determine if the deflation parameter has been met by inspection or observation. In some embodiments, deflation parameter detection may be instrumented using one or more sensors. For example, monitoring the parameter indicative of a degree of inflation of cuff 125 in step 1510 may include monitoring the inflation pressure within cuff 125 using a pressure sensor such as a manometer or the like (e.g. inflation pressure sensor 151B). Alternatively/additionally where a gas flow is being provided to the patient during step 1510, said step 1510 may include monitoring a gas outflow between the patient's airway and the invasive patient interface 120 wherein a flow rate greater than zero indicates that the first exit flow path 270A has formed. In some embodiments, an outflow rate in the first exit flow path 270A of at least about 0.5 LPM, or about 5 LPM or in some embodiments, about 20 LPM indicates that cuff 125 has been deflated enough to provide the desired non-sealing arrangement and the majority of outflow is in the first exit flow path 270A. Alternatively/additionally said step 1510 may include monitoring the rate of outflow within the invasive patient interface 120, i.e. in the second exit flow path 270B. In some embodiments, an outflow rate in the second exit flow path 270B of less than the rate at which flow is provided into the invasive patient interface 120 indicates that cuff 125 has been deflated enough to provide the non-sealing arrangement. An outflow rate in the second exit flow path 270B is less than the inflow rate into the invasive patient interface 120 because some of outflow is in the first exit flow path 270A. Alternatively/additionally said step 1510 may include monitoring the velocity and/or flow rate of gas exiting the proximal end of the invasive patient interface 120. In some embodiments, an increase in the velocity and/or exit flow rate of gases out of the invasive patient interface indicates that cuff 125 has been deflated enough to provide the non-sealing arrangement which provides a reduction in the resistance to flow sufficient to achieve the increase in exit velocity and/or flow rate. Alternatively/additionally, said step 1510 may include monitoring pressure in the second exit flow path 270B. In some embodiments, a reduction in the pressure in the second exit flow path 270B indicates that cuff 125 has been deflated enough to provide the non-sealing arrangement which permits gases to also exit via the first exit flow path 270A. Conversely, an increase in the pressure in the second exit flow path 270B indicates that cuff 125 has been increased enough to provide the sealing arrangement which prevents gases from exiting via the first exit flow path 270A. Alternatively/additionally, said step 1510 may include monitoring CO₂ in the second exit flow path 270B. In some embodiments, a reduction in the CO₂ in the second exit flow path 270B indicates that cuff 125 has been deflated enough to provide the non-sealing arrangement which permits expired gas and hence at least a portion of the CO₂ exiting the patient to also exit via the first exit flow path 270A. Conversely, an increase in the CO₂ in the second exit flow path 270B indicates that cuff 125 has been inflated enough to provide the sealing arrangement which prevents expired gas (and the CO₂ exiting the patient) from exiting via the first exit flow path 270A.

Alternatively/additionally it may be desirable to monitor the distance between the outer wall of the cuff 125 from the inner wall of the airway 310 to ensure the cuff has been deflated sufficiently that the two are separated by at least a predetermined distance at the sealable member. This may be achieved using ultrasound, endoscope or other imaging or detection means to identify the landmarks required to determine the predetermined distance. Alternatively/additionally it may be desirable to monitor the cross sectional area of the deflated cuff 125, relative to the cross sectional area of the airway and/or the cross sectional area of the region between the invasive patient interface and the patient's airway, and determine that the non-sealing arrangement has been achieved when the cross sectional area of the deflated cuff 125 corresponds to less than 100% occlusion of the region between the invasive patient interface and the patient's airway.

Typically, prior to step 1100, patient 300 is induced into a state of general anaesthesia in a step 1010 so that the patient is no longer spontaneously breathing. Normally the patient is spontaneously breathing prior to induction of anaesthesia although that need not always be the case. Induction of anaesthesia may be achieved by a clinician or anesthetist introducing a drug such as propofol into the patient's circulation by injection or intravenous line, or by delivering anaesthetic gases such as desflurane, isoflurane or sevoflurane which are inhaled through a mask placed over the patient's nose and mouth.

In some embodiments, method 1000 includes in a step 1500 detecting absence of spontaneous breathing by the patient prior to intubating the patient in step 1100. Detection of the absence/presence of spontaneous breathing can be achieved by inspection or observation of the patient by a clinician or other attendant. When absence of spontaneous breathing is detected, intubation of the patient commences. Upon or after successful intubation, the cuff 125 may be operated by the clinician or other attendant using inflation means 155. Operation of cuff 125 includes but is not limited to maintaining deflation of a deflated cuff and deflating an inflated cuff. It may be beneficial to inflate the cuff 125 upon successful intubation to provide the patient with a second respiratory support, e.g. mechanical ventilation for purposes such as pressure support, before deflating the cuff 125 to provide the patient with a first respiratory support, e.g. continuous flow during respiratory stillness. This could also provide the added benefit of allowing a clinician or user to configure the non-sealing arrangement before providing the first respiratory support.

In some embodiments, the clinician or other attendant may provide input via user input device 152 to a controller 150 indicating that the patient is not spontaneously breathing, whereby controller 150 operates or causes operation of cuff 125 using inflation means 155. Alternatively/additionally, controller 150 may receive input by other means involving use of one or more sensors such as respiratory, motion or other suitable sensors confirming that the patient is not spontaneously breathing. It is desirable to check for unconsciousness and/or the absence of spontaneous breathing by the patient at step 1500 prior to intubating the patient at 1100. After successful intubation a non-sealing arrangement can be achieved and a flow of respiratory gas can be provided to the patient at 1300 to ensure the patient is provided with adequate respiratory support in a medical procedure that requires substantial stillness of the patient particularly in the area of the thorax and/or abdomen, or at least a form of respiratory support that itself does not cause movement of the patient. Provision of respiratory support according to method 1000 (and method 3000 discussed below) can occur while the patient is apnoeic, i.e. while there is no spontaneous breathing or provision of mechanical ventilatory support. Advantageously, provision of respiratory support according to method 1000 does not cause displacement of the patient's diaphragm, thorax and/or abdomen, and therefore supports this objective. Advantageously, provision of respiratory support according to method 1000 promotes a safe and prolonged period (or periods) of respiratory stillness.

Following delivery of respiratory gas according to step 1300, the clinician or other operator may cause the cuff to be inflated. Alternatively, the clinician or other operator may provide an input to the controller 150 that the form of respiratory support is to be switched, and the controller will respond by causing inflation of the cuff. Both will cause switching of the respiratory support upon detection by the controller 150 (or observation by a user who provides an input to the controller) that the cuff is in the inflated state, after which the respiratory support becomes dependent on an inspiratory and expiratory intended breathing cycle of the patient. Subsequent changes in cuff state (i.e. deflation/inflation) and switching between modes of respiratory support may follow as may be selected by a clinician or other operator to provide the necessary respiratory support to the patient.

FIG. 7 is a schematic illustration of an exemplary connector 200 for providing a flow of respiratory gas according to embodiments of systems and methods disclosed herein. Connector 200 includes a main body 210 having a gases port 220 for receiving a flow of gas from the flow source 110, an outlet port 230 for outflow of gases from the main body 210, and a device port 240 couplable with the invasive patient interface 120. The gases port 220 includes an inlet 216 and an outlet 260 which may include one or more constrictions and in the embodiment shown, includes a nozzle 260. The connector 200 is configured to receive the flow of gas from the flow source 110 via the inlet 216 of the gases port 220, and to provide a flow of gas through the outlet 260. This arrangement may achieve a predetermined patient pressure in the airway during use between about 2 cmH₂O and about 20 cmH₂O, or about 2 cmH₂O to about 10 cmH₂O, or about 2 cmH₂O and about 5 cmH₂O, or about 5 cmH₂O to about 10 cmH₂O. at flow rates received from the flow source of about 0 LPM to about 150 LPM, preferably about 20 LPM to about 70 LPM. A pressure loss between the device port 240 and the outlet port 230 of the connector 200 is less than about 20 cmH₂O when in use.

In the embodiment shown, a flow constriction 250 is in the form of a tapered nozzle 260 extending into the main body 210 of the connector 200. The connector 200 also includes an outlet port 230 with an outlet channel 232 that forms part of the exit flow path 270B through which gases exit from the connector 200. The exit flow path 270B in the connector 200 is defined between the device port 240 and the outlet port 230. The outlet port 230 may be oriented in a variety of ways, with respect to connector body 210. In the embodiment shown, outlet port 230 is oriented to provide an offset outlet channel 232. The offset is at an obtuse angle relative to a longitudinal axis of the inlet channel 222 such that the outlet port 230 is adjacent the device port 240. In other embodiments (not shown) the connector may include an offset at an acute angle such that the outlet port 230 is adjacent the gases port 220.

Parameters of connector 200 may be adjusted to influence characteristics of gas flow provided through the connector to the patient's airway. Firstly, outlet 260 of the tapered nozzle has a diameter (which may determine cross-sectional area) denoted as parameter A. Secondly, there is a desired distance, denoted as parameter B, between the outlet 260 and a proximal end opening 242 of the device port 240. Thirdly, there is a minimum diameter (which may determine cross-sectional area) of the expiratory path 270, denoted as parameter C (notably this varies according to location within the outlet channel 232 as well as the varying offset of the outlet channel 232 and nozzle alignment). These parameters permit optimisation of the connector's performance to achieve certain objectives such as, for example, a predetermined patient pressure, a predetermined flow velocity, and a range of flows that can be provided to the patient at the patient pressure and/or predetermined velocity. Preferably, the flow of gas from outlet 260 includes a velocity in a range of up to about 60 m/s, or up to about 30 m/s, preferably at least about 5 m/s, and in some embodiments preferably at least about 10 m/s, preferably at a selected flow rate of about 20 L/min to about 70 L/min of the flow of gas provided by the flow source 110. Details concerning selection of parameters A, B and C for a range of connectors 200 are disclosed in detail in commonly owned U.S. Provisional Patent Application 63/079,651 filed 17 Sep. 2020, which is hereby incorporated herein by reference. It is to be understood that while the constrictions are shown within the connector body 210 in FIG. 7, in some embodiments a lumen may be provided e.g. with or as part of the connector, which extends within the invasive patient interface 120 and provides a constriction in the form of an outlet near the proximal tip of the invasive patient interface. The outlet includes but is not limited to an outlet of a catheter inserted in the invasive patient interface, and an outlet of a flow path formed between a catheter inserted in the invasive patient interface and the invasive patient interface itself. The lumen parameters may be optimised to achieve certain objectives such as, for example, a predetermined flow velocity which may be provided at a target location such as near the carina or deeper in one or more bronchi and/or bronchioles.

Optional gas sampling ports 214, 234 are also shown, for sampling one or more characteristics of the gases in the main body 210 of the connector 200. The one or more characteristics of the gases sampled at the ports 214, 234 may include pressure, flow rate, concentration, gas constituents (e.g., oxygen and carbon dioxide), temperature, humidity, contaminants, aerosols and/or pathogens. The connector 200 may include one or more gas sampling ports 234 located on the outlet port 230, such as for sampling levels of contaminants, aerosols and/or pathogens located in the gases exiting the main body 210. FIG. 7 shows a single gas sampling port 234 on the outlet port 230, although some embodiments may include a plurality of ports, and the one or more ports may be located at any location along a length of the outlet port 230 having the outlet channel 232. As shown in FIG. 7, the gas sampling port 214 may be located near outlet 260. Although not shown, the gas sampling port 214 may be located along a length of the device port 240. FIG. 7 shows a single gas sampling port 214, although embodiments may include a plurality of ports as would be appreciated by a person skilled in the art, for sampling one or more characteristics of gases at various points in the connector 200.

In some embodiments, the connector 200 may also include a suctioning port (not shown). The suctioning port may be located in the same or similar positions on the connector main body 210 or outlet port 230 of the connector 200 as the gas sampling ports 214, 234. The suctioning port may provide for removal of bodily fluids from the connector 200, such as mucus.

### Switching Between Forms of Respiratory Support

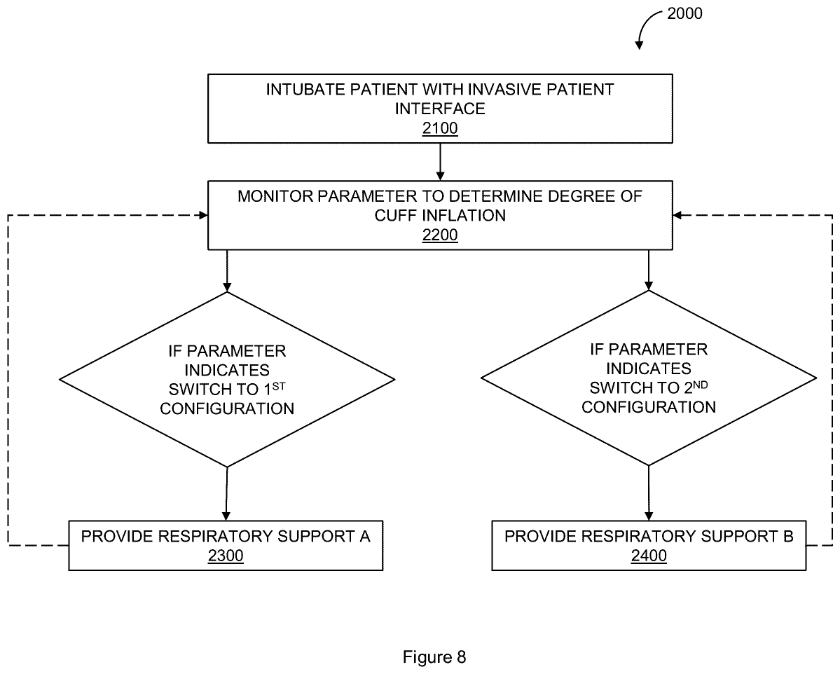

Referring to FIG. 8, a flow chart shows in schematic form steps in a method 2000 for providing respiratory support to a patient according to an embodiment of the disclosure involving switching between different forms of respiratory support. In a step 2100 patient 300 is intubated with an invasive patient interface 120 having a sealable member 125 (FIG. 3). Invasive patient interface 120 is operable in a first configuration or a second configuration. According to method 2000, one form of respiratory support (Respiratory Support A) is provided when the invasive patent interface is in one configuration (1^{ST} configuration) and responsive to the invasive patient interface being in the other configuration (2^{ND} configuration), the respiratory support being provided is switched to a different form of respiratory support (Respiratory Support B). It is to be noted that although not shown in the figure, following initial intubation of the patient at step 2100, a clinician or other operator may select the form of respiratory support to be provided (Respiratory Support A or Respiratory Support B) after which inflation or deflation of the cuff will follow, or they may select the first or second configuration for the inflatable cuff, after which provision of Respiratory Support A or Respiratory Support B will follow. Selection may be performed by providing an input to the controller 150 or directly altering the inflation configuration of the cuff. In some embodiments, the extent of cuff deflation may be determined based on clinical therapy judgement which is performed either by the clinician or other operator, or by the controller based on input received from the clinician or other operator and/or sensors.

In other embodiments (not shown) it may be desirable to switch forms of respiratory support and, in response to a change in a parameter which is indicative of the change in respiratory support being provided to the patient, alter the arrangement of the sealable member within the airway. Thus, in some embodiments, a method for providing respiratory support to a patient may involve, responsive to the respiratory support changing from one form to a different form, changing the configuration of the invasive patient interface. Thus, responsive to a change to provide Respiratory Support A, the sealable member is altered to adopt the first configuration, and responsive to a change to provide Respiratory Support B, the sealable member is altered to adopt the second configuration. Alteration of the condition of the sealable member may be automated by a controller, or it may be achieved manually by a clinician or other attendant, e.g. by inflating or deflating an inflatable balloon or cuff which comprises the sealable member.

In some embodiments, in the first configuration the sealing member 125 forms a non-sealing arrangement of the invasive patient interface within the patient's airway as shown in FIG. 12f, and in the second configuration the sealing member forms a sealing engagement between the invasive patient interface 125 and the patient's airway 310 as shown in FIG. 12g (right illustration). A clinician may determine a switch or change in the configuration of the invasive patient interface by inspection and/or observing/monitoring the state of inflation/deflation of an inflatable cuff comprising the sealable member 125 and switch the form of respiratory support being provided accordingly. In some embodiments this involves switching between modes of support provided by a single flow source, or coupling and/or decoupling one or more separate flow sources by use of an interchangeable connector such as e.g. wye pieces 185A/185B and connector 200 as discussed below. In some embodiments, physical apparatus including sensors and a controller 150 may be utilised to determine a switch or change in the configuration of the invasive patient interface 120 and optionally to alter the form of respiratory support being provided accordingly. It is to be understood that in some embodiments, the respiratory support being provided may be monitored, and upon observation or detection of a switch or change in the form of respiratory support being provided, the configuration of the invasive patient interface may be switched accordingly, either manually or under control of controller 150.

In some embodiments method 2000 includes, after intubating the patient, in a step 2200 monitoring a parameter to determine a degree of inflation of cuff 125 to determine if it is in the first configuration i.e. in non-sealing arrangement within the patient's airway or in the second configuration, i.e. in a sealing arrangement within the patient's airway. If the parameter indicates the first configuration, Respiratory Support A is provided in step 2300. If the parameter indicates the second configuration, Respiratory Support B is provided in step 2400. The parameter may be monitored continuously or intermittently during provision of respiratory support as represented by broken lines in FIG. 8. The parameter may be monitored by instrumented input from sensors to a controller, or it may be monitored by clinical observation, wherein a clinician or other attendant monitors the condition or degree of inflation of cuff 125 and manually adjusts the respiratory support according to the observed condition/degree of inflation.

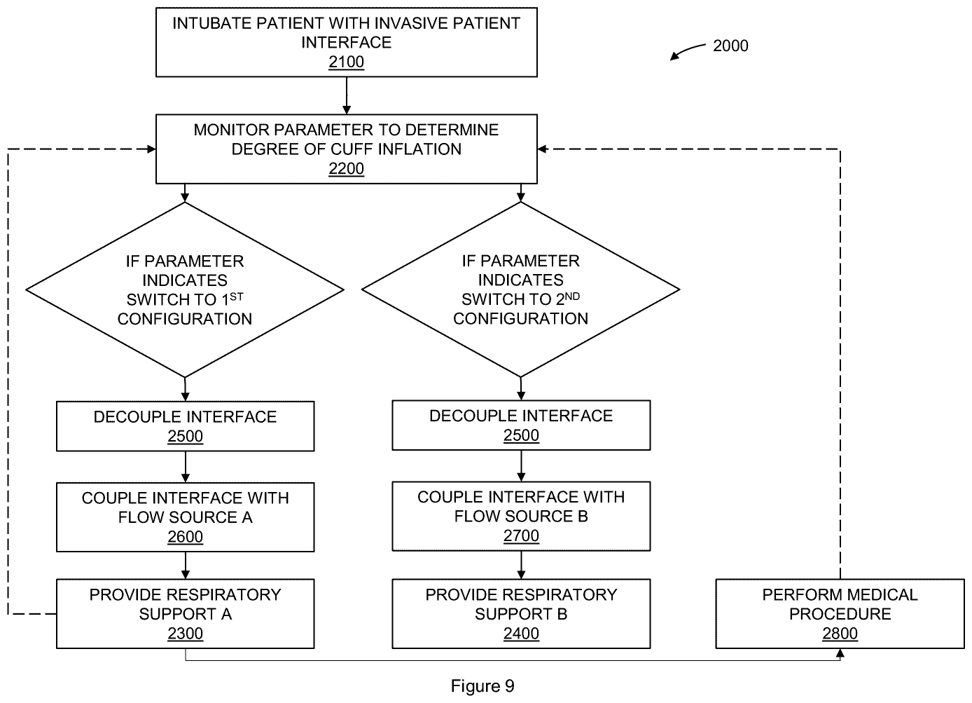

Monitoring the parameter indicative of a degree of inflation/deflation of cuff 125 in step 2200 may include one or more of monitoring the inflation pressure within cuff 125, monitoring rate of outflow around the invasive patient interface 120, monitoring the rate of outflow within the invasive patient interface 120, as described in the context of method 1000 which is hereby explicitly stated to be relevant to the method 2000, or using other techniques as may be understood by one of skill in the art. The parameter may be monitored substantially continuously during provision of respiratory support as represented by broken lines in FIG. 8 and FIG. 9, or monitored intermittently, for example.

In some embodiments, Respiratory Support A which is provided when the invasive patent interface is in the first configuration comprises a substantially continuous flow of gas and may be independent of spontaneous or non-spontaneous (i.e. artificially ventilated) inspiratory or expiratory breathing cycles of the patient. Respiratory Support A may be flow controlled, where a gas flow is controlled to a desired flow rate. In some embodiments, Respiratory Support A may be flow controlled respiratory support which provides a gas flow having a flow rate in a range of about 0 LPM to about 120 LPM, preferably about 20 LPM to about 70 LPM and in some embodiments, at least about 40 LPM. In some embodiments the flow rate is variable.

In contrast, Respiratory Support B which is provided when the invasive patent interface is in the second configuration comprises a cyclic flow of gas providing inspiratory and expiratory phases. In some embodiments, Respiratory Support B may be dependent on an intended breathing cycle of the patient. For example, Respiratory Support B may comprise a pressure and/or volume controlled flow of respiratory gas providing respiratory support having a pressure and/or volume wave form that somewhat resembles cycles of spontaneous breathing. In some embodiments Respiratory Support B comprises mechanical or manual ventilation providing inspiratory and expiratory phases of gas flow within the patient's airway and may be provided by a ventilator or an anaesthesia machine. Manual bag ventilation is also contemplated. Alternatively, Respiratory Support B which is provided when the invasive patent interface is in the second configuration may comprise a non-cyclic flow of gas such as a continuous or oscillating flow of gas which may be flow or pressure controlled, akin to Respiratory Support A.

In some embodiments, each of Respiratory Support A and Respiratory Support B comprises a flow of respiratory gas originating from a common flow source 110. In other embodiments, Respiratory Support A and Respiratory Support B comprises a flow of respiratory gas originating from separate flow sources 110, 112 (FIG. 6) which are coupled and decoupled with the invasive patient interface 120 as necessary. FIG. 9 is a flow chart showing in schematic form steps in a modified version of method 2000 in which Respiratory Support A is provided by a flow source A and Respiratory Support B is provided by a flow source B. It is to be noted that although not shown in the figure, following initial intubation of the patient at step 2100, a clinician or other operator may select the form of respiratory support to be provided (Respiratory Support A or Respiratory Support B) after which inflation or deflation of the cuff will follow, or they may select the first or second configuration for the inflatable cuff, after which provision of Respiratory Support A or Respiratory Support B will follow. Selection may be performed by providing an input to the controller 150 or directly altering the inflation configuration of the cuff. In some embodiments, the extent of cuff deflation may be determined based on clinical therapy judgement which is performed either by the clinician or other operator, or by the controller based on input received from the clinician or other operator and/or sensors.

In this embodiment, the method may further include, in a step 2500, decoupling the invasive patient interface 120 from an already connected flow source, and in a step 2600 coupling the invasive patient interface with flow source A when there is a change/switch to the first configuration, or in a step 2700 coupling the invasive patient interface with flow source B when there is a change/switch to the second configuration. Flow source A may be configured to provide a substantially continuous flow of respiratory gas. Flow source B may be configured to provide a cyclic and/or pressure and/or volume controlled flow of respiratory gas.

One or both of Respiratory Support A and Respiratory Support B may include humidifying the respiratory gas. In some embodiments, at least Respiratory Support A includes humidifying the respiratory gas. Typically, one or both of Respiratory Support A and Respiratory Support B are provided during absence of spontaneous breathing by the patient. Switching between forms of respiratory support may be achieved as many times as may be desired by a clinician or attendant responsible for the patient's respiratory support.

The method 2000 may further include during provision of Respiratory Support A, in a step 2800 performing a medical procedure that benefits from little or no movement of the patient due to respiratory gas exchange. The form of respiratory support being provided can be switched between Respiratory Support A and Respiratory support B by changing the configuration of the invasive patient interface and switching the form of respiratory support accordingly. Where the different forms of respiratory support originate from different flow sources, the method includes coupling and decoupling the invasive patient interface between flow sources as necessary to provide the relevant support. While Respiratory Support A can be provided to the patient to prolong a safe period of respiratory stillness, the method 2000 permits switching between Respiratory Support A and Respiratory Support B which may be desired in circumstances where it may be desirable to e.g. improve CO₂ clearance by providing one or more cycles of ventilation (pressure and/or volume controlled inspiratory and expiratory flows of respiratory gas), providing pressure, delivering volatile agents to the patient to extend duration of anaesthesia, and reducing risk of gastric aspiration.

FIG. 6 is a schematic illustration of a system 100 with a controller 150, for providing respiratory support to a patient 300 during a medical procedure. The system comprises a flow source 110 operable to provide a flow of gas for provision to the patient via an invasive patient interface 120 and a controller 150 receiving one or more inputs and operable to control the flow source according to one or more received inputs. System 100 is couplable with an invasive patient interface 120 having a sealable member 125 that is operable to achieve a first system condition in which there is non-sealing arrangement of the invasive patient interface within the patient's airway (FIG. 12g left illustration), and a second system condition in which there is sealing engagement with the patient's airway (FIG. 12g right illustration). Controller 150 controls flow source 110 to provide a first respiratory support in the first system condition and a second respiratory support in the second system condition.

In some embodiments, the controller may include a rotary switch 111 operable to control flow characteristics of respiratory gas provided in the respiratory support. In this arrangement, rotary switch 111 is used to control characteristics of the first respiratory support provided in the first system condition. In some embodiments, system 100 includes a controller 150 which has functionality or an associated element which permits control of the flow source to provide the second respiratory support when in the second configuration.

In some embodiments, controller 150 controls flow source 110 such that the first respiratory support is independent of an inspiratory and expiratory breathing cycle of the patient, and/or the second respiratory support is dependent on or imitates an inspiratory and expiratory breathing cycle of the patient which may be spontaneous or non-spontaneous. Thus, the first respiratory support may comprise a substantially continuous flow of gas and/or the second respiratory support may be a cyclic flow of gas providing inspiratory and expiratory phases. In some embodiments system 100 comprises a single flow source 110 operable to provide a continuous flow of gas for providing the first respiratory support as well as a cyclic flow of gas having inspiratory and expiratory phases for providing the second respiratory support. Thus, the single flow source 110 may comprise a flow generator or blower capable of generating a gas flows which may be flow controlled (e.g. generating flow rates of about 0 LPM to about 150 LPM, preferably about 20 LPM to about 70 LPM and in some embodiments, preferably at least about 40 LPM) and/or pressure and/or volume controlled (e.g. generating cyclic flows of gas for lung inflation and deflation) under the control of controller 150. In embodiments where flow source 110 includes a blower, increasing or decreasing the flow rate may be achieved by the controller e.g. increasing or decreasing the motor speed of the blower. Alternatively/additionally the controller may control operation of a proportional valve of the flow source 110 to produce pressure and/or tidal volume waveforms and/or flows for provision of the required respiratory support.

In other embodiments, flow source 110 comprises a first flow source 110 providing a substantially continuous flow of gas and a second flow source 112 providing a cyclic flow of gas having inspiratory and expiratory phases. The first and second flow sources 110, 112 may be integrated into a single apparatus or they may be separate apparatus (as shown) which operate under control of controller 150 which controls the first and second flow sources 110, 112 according to inputs received which are indicative of the system 100 being in the first system condition (in which respiratory gas is provided from first flow source 110) or the second system condition (in which respiratory gas is provided from second flow source 112).

Irrespective of whether system 100 comprises a single flow source 110 or a first and second flow sources 110, 112, controller 150 may be operable to control the rate of flow of gas provided for provision of the first respiratory support. Controller 150 may provide variable control of the rate of flow of gas. Controller 150 may control the rate of flow of gas in a range of 0 to about 150 LPM, preferably about 20 LPM to about 70 LPM and in some embodiments, at least about 40 LPM. Furthermore, controller 150 may be operatively coupled with the first flow source 110 and/or second flow source 112 by wired or wireless means as would be understood by one of skill in the art.

Irrespective of whether system 100 comprises a single flow source 110 or a first and second flow source 110,112, controller 150 may be operable to control pressure and/or volume and/or flow of respiratory gas provided by the flow source 110/112 to the patient for provision of the second respiratory support. Controller 150 may be operable to control the pressure and/or volume substantially in synchrony with inspiratory and expiratory phases of a breathing cycle intended for patient 300, or to mimic or induce these breathing cycles.

Irrespective of whether system 100 comprises a single flow source 110 or a first and second flow source 110, 112, controller 150 may be operable to control the flow source/s to provide a flow of gas at a target location deep in the patient's airway such as near or at the carina or deeper in one or more bronchi and/or bronchioles at a predetermined velocity e.g. in a range of up to about 60 m/s, or up to about 30 m/s, preferably at least about 5 m/s, and in some embodiments preferably at least about 10 m/s. Controller 150 may provide variable control of the predetermined velocity.

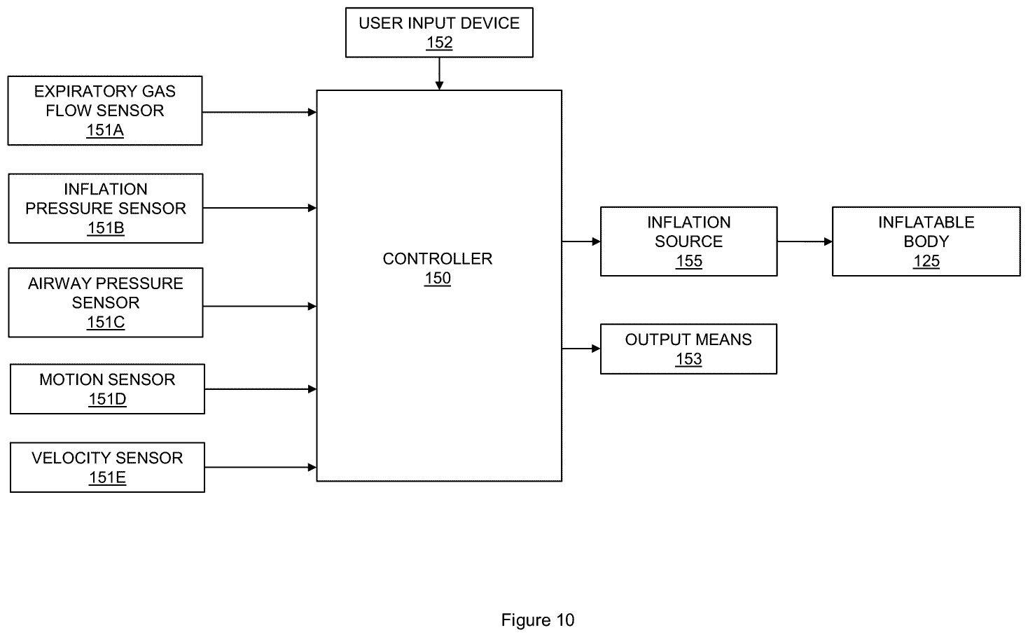

FIG. 10 is a schematic illustration showing controller 150 which is configured to receive one or more inputs. In some embodiments the inputs are used to determine e.g. if the system is in the first system condition or the second system condition. The inputs may be received from a user who enters the designation or selection invoking use of the system in the first system condition or second system condition using a user input device 152. Alternatively/additionally, inputs receivable by controller 150 may comprise signals generated by one or more sensors 151A-D detecting a parameter used to determine if the system is already in the first system condition or the second system condition. Inputs receivable by controller 150 may comprise signals generated by an expiratory gas flow sensor 151A (e.g. which measures a flow rate, pressure and/or gas concentration in the patient's airways, respiratory system and/or patient interface, e.g. sealable member 125) and/or an inflation pressure sensor 151B associated with an inflatable body comprising the sealable member 125, an airway pressure sensor 151C, a motion sensor 151D (e.g. which monitors patient's chest movements). Inputs may be receivable by controller 150 by wired or wireless means, as would be appreciated by one of skill in the art.

In some embodiments, an expiratory gas flow sensor 151A may monitor flows of exiting gases inside the invasive patient interface. This may be achieved by direct measurement using a gas flow sensor located in or fed by a sampling line in the invasive patient interface, or by indirect measurement using a gas flow sensor located in or fed by gas samples from e.g. a connector coupled with the invasive patient interface. Indirect measurements may require adjustment for flow characteristics not related solely to flows inside the invasive patient interface. Controller 150 may determine a lower expiratory gas flow rate inside the invasive patient interface 120 e.g. lower than the rate at which gas flow is provided into the invasive patient interface to indicate there is gas flow through the patient interface and out through exit flow path 270A between the invasive patient interface and the patient's airway, indicating that the invasive patient interface is non-sealingly engaged within the airway and the system is in the first system condition. Conversely, controller 150 may determine an expiratory outflow rate inside the invasive patient interface 120 which is about equivalent to the rate at which gas flow is provided to the invasive patient interface which indicates that exit flow path 270A may be absent and that sealable member 125 is in sealing engagement with the patient's airway, and the system is in the second system condition.

Alternatively/additionally, an expiratory gas flow sensor 151A may monitor outflows of gases near an external wall of invasive patient interface 120. Controller 150 may determine a gas outflow rate outside the invasive patient interface 120 e.g. greater than 0 LPM, or at least about 0.5 LPM, or about 5 LPM or in some embodiments, about 20 LPM to indicate gas outflow from airway 310 is around the invasive patient interface, indicating that the invasive patient interface is non-sealingly arranged within the airway and the system is in the first system condition. Conversely, controller 150 may determine substantially zero expiratory outflow rate outside the invasive patient interface 120 to indicate gas outflow from airway 310 is via the invasive patient interface 120, and not around it, indicating that sealable member 125 is in sealing engagement with the patient's airway and the system is in the second system condition. In other words, expiratory gas flow sensors 151A may provide an indication as to whether exit flow path 270A is formed or not.

Alternatively/additionally, controller 150 may determine a low pressure within the inflatable body, e.g. lower than 30 cmH₂O, preferably lower than 25 cmH₂O and more preferably lower than 20 cmH₂O to indicate invasive patient interface 120 is in non-sealing arrangement within the patient's airway 310 and the system is in the first system condition. In some embodiments a lower predetermined inflation pressure may be preferred to increase CO₂ clearance. In some embodiments, an inflation pressure of about minus 10 cmH₂O relative to atmosphere can achieve effective CO₂ clearance. Conversely, controller 150 may determine a higher pressure within the inflatable body e.g. higher than 20 cmH₂O, preferably higher than 25 cmH₂O to indicate the invasive patient interface 120 is sealingly engaged with the patient's airway and the system is in the second system condition.

In some embodiments, system 100 includes a connector 200 configured to provide a flow of gas through outlet 260 at, at least, a predetermined flow rate or a predetermined velocity, and in some embodiments, at a predetermined patient pressure. A schematic illustration of one such connector is provided in FIG. 7 and other examples of suitable connectors are described in commonly owned U.S. Provisional Patent Application 63/079,651 filed 17 Sep. 2020, which is hereby incorporated herein by reference.

The characteristics of the flow of gas is provided by a connector 200 is a result of parameters of connector including the diameter (alternatively denoted as cross sectional area A) at outlet 260, a distance B between outlet 260 and opening 242, and diameter of the expiratory path through gases exit port 230 (alternatively denoted as cross sectional area C) as well as the flow rate of gas received into gases port 220 and the internal diameter of invasive patient interface 120. While a single nozzle outlet 260 is shown in FIG. 7, the connector may comprise a plurality of apertures or constrictions for generating an outflow velocity through outlet 260. The outflow velocity preferably includes a velocity that is capable of achieving at least a predetermined airway pressure of at least 2 cmH₂O when in use. Preferably, the outflow velocity through outlet 260 is greater than the velocity of a gases flow provided or generated by flow source 110. Preferably, the outflow velocity through outlet 260 includes a velocity in a range of about 5 m/s to about 60 m/s. Preferably, the outflow velocity through outlet 260 includes a velocity in a range of about 5 m/s to about 60 m/s at a selected flow rate of about 20 L/min to about 70 L/min of the flow of gas provided by the flow source 110. In some embodiments, the outflow velocity through outlet 260 may contribute to achieving the predetermined velocity at a target location deep in the patient's airway such as at or near the carina or deeper in one or more bronchi and/or bronchioles.

In some embodiments system 100 comprises a gas delivery conduit 180 between flow source 110 and connector 200, wherein the connector, when coupled with the invasive patient interface 120, provides a flow path for delivery of the first respiratory support to the patient. In some embodiments, connector 200 may be integral with the delivery conduit 180. This may be desirable in embodiments where the flow source comprises separate first and second flow sources 110, 112 and a designated delivery conduit 180 is integral with connector 200 facilitating easy decoupling and coupling of the connector 200 with the invasive patient interface 120 when switching between the first respiratory support and the second respiratory support.

In some embodiments, system 100 includes means for operating the sealable member to achieve sealing engagement with the patient's airway or a non-sealing engagement of the invasive patient interface within the patient's airway. Typically, the sealable member is an inflatable body or balloon in the form of cuff 125 which may be inflated by e.g. a syringe pump 155 (or a bulb or other pump) which is described in relation to FIG. 5. Although not shown in FIG. 5, it is to be understood that the pump or other inflation source 155 provided to achieve inflation or deflation of the sealable member 125 may be operatively coupled with controller 150 so that the controller controls the inflation source to inflate the inflatable body to an inflation pressure sufficient for achieving sealing engagement with the patient's airway, consistent with the second system condition. Similarly, controller 150 may control inflation source 155 to deflate the inflatable body to a predetermined inflation pressure (or less) to achieve non-sealing engagement of the invasive patient interface within the patient's airway, consistent with the first system condition. The predetermined inflation pressure is less than a sealing pressure required to achieve sealing engagement with the patient's airway and is typically less than about 20 cmH₂O to about 30 cmH₂O. In some embodiments, a preferred predetermined inflation pressure is about minus 10 cmH₂O relative to atmosphere.

Alternatively/additionally, controller 150 may cause deflation of the inflatable body 125 to occupy a cross sectional area corresponding to less than 100% occlusion of the region between the invasive patient interface and the patient's airway.

As represented schematically in FIG. 10, system 100 may include a range of sensors 151 including flow sensor 151A, pressure sensors 151B, 151C, and motion sensor 151D and gas flow velocity sensor 151E which may be used to determine system condition. Flow sensor 151A may be a flow rate sensor. In some embodiments, controller 150 may receive inputs from a motion sensor 151D configured to detect movement of the patient during provision of the respiratory support, and wherein the controller generates a control signal to produce an audible and/or visible and/or haptic output indicating substantial absence of movement during provision of respiratory support by the system. The motion sensor 151D may also provide a signal indicative of presence/absence of spontaneous breathing if placed on the chest, since cyclic rise and fall indicates inflation and deflation of the lungs, which in the absence of artificial ventilation, is consistent with spontaneous breathing. Motion sensor 151D may include an accelerometer, gyroscope, pressure triggered sensor or other sensor configured to detect movement, typically of the abdomen and/or thorax, as would be known to one of skill in the art.

In some embodiments, system 100 includes output means 153 which is operable by controller 150 to provide an output that is discernible to a clinician or other attendant. Output means 153 may include one or more of a visual display device or monitor, a speaker for providing audible signals and a haptic feedback module providing tactile feedback concerning one or more of the system being in the first system condition or the second system condition, the presence/absence of spontaneous breathing by the patient and the like.

### Respiratory Support During a Medical Procedure

In another embodiment, system 100 is configured for providing respiratory support to a patient 300 during a medical procedure, wherein a controller controls the flow source to provide the required form of respiratory support. System 100 comprises a flow source 110 configurable to provide a flow of gas for delivery to the patient via an invasive patient interface 120 and a controller 150 controls the flow source. The system is couplable with, or includes, invasive patient interface 120 which has sealable member 125 that is configurable to form a sealing engagement with the patient's airway. In response to invasive patient interface 120 being non-sealingly arranged within the patient's airway during absence of spontaneous breathing, controller 150 controls the flow source 110 to provide a flow of gas for delivery to the patient. The respiratory support provided preferably maintains a period of safe apnea or respiratory stillness while the invasive patient interface is non-sealingly arranged within the patient's airway.

In this arrangement, the flow of gas provided by the flow source 110 is substantially continuous, albeit time-varying (e.g. oscillatory). The substantially continuous flow may be a unidirectional or positive net flow towards the patient. Furthermore, the selected flow rate for the flow source 110 may be a fixed flow rate, which may be variable upon input to the controller 150 to vary the rate. The fixed flow rate may be independent of the respiratory cycle of the patient 300. The flow rate may be in a range of 0 LPM to about 150 LPM, preferably about 20 LPM to about 70 LPM.

Controller 150 may be configured to receive one or more inputs 157 and to control flow source 110 according to the one or more received inputs. In some embodiments, controller 150 is configured to receive one or more inputs 157 that are indicative of e.g. arrangement of the invasive patient interface within the patient's airway and/or presence/absence of spontaneous breathing by the patient.

Inputs 157 received by controller 150 comprise signals generated by one or more sensors, such as an exiting gases flow sensor, a pressure sensor associated with an inflatable body comprising the sealable member and an airway pressure sensor as previously described herein, or inputs received via a user input device 152 corresponding to the desired control of elements of the system, e.g. inflation or deflation of the cuff 125, or selection of the form of respiratory support required to be provided to the patient. By way of non-limiting example, FIG. 6 shows exiting gases flow sensor 151A located on invasive patient interface 120 for detecting flow rates of gas in first exit flow path 270A outside invasive patient interface 120. Controller 150 may determine a gas outflow rate outside the invasive patient interface 120 e.g. higher than 0 LPM to indicate gas outflow from airway 310 is around the invasive patient interface and not through it, indicating that the invasive patient interface is non-sealingly arranged within the airway. Conversely, controller 150 may determine a very low or zero gas outflow rate outside the invasive patient interface 120 to indicate gas outflow from airway 310 is via the invasive patient interface 120, and not around it, indicating that sealable member 125 is in sealing engagement with the patient's airway.

Alternatively/additionally, a pressure sensor such as a manometer or the like may provide input to controller 150 indicating inflation pressure within inflatable body 125. An inflation pressure less than a predetermined inflation pressure of e.g. 30 cmH₂O, preferably less than 25 cmH₂O and more preferably less than 20 cmH₂O may indicate invasive patient interface 120 is in non-sealing arrangement within the patient's airway 310. In some embodiments, a preferred predetermined inflation pressure is about minus 10 cmH₂O relative to atmosphere.

Conversely, controller 150 may determine a higher pressure within the inflatable body 125 e.g. higher than about 20 cmH₂O, preferably higher than about 25 cmH₂O or about 30 cmH₂O to indicate the invasive patient interface 120 is sealingly engaged with the patient's airway.

Alternatively/additionally, a pressure sensor detecting patient airway pressure may provide input to controller 150. An airway pressure less than the airway pressure measured when there is sealing engagement may indicate that invasive patient interface 120 is in non-sealing arrangement within the patient's airway 310.

In some embodiments, system 100 is operable to provide an alternative form of respiratory support, responsive to the controller 150 determining that the invasive patient interface is in or has switched to a condition in which it is sealingly engaged with the patient's airway 310. This is achieved when the sealable member or inflatable body/balloon/cuff is inflated to form sealing engagement with the airway 310. Controller 150 may receive an input indicative of the sealing engagement via user input device 152 or a sensor providing an input 157 to the controller from which it can be determined that there is sealing engagement. When such an input is received, controller 150 controls flow source 110 to provide the alternative form of respiratory support which in some embodiments, includes a cyclic flow of respiratory gas. The alternative form of respiratory support may be dependent on an intended breathing cycle of the patient, providing or following a desired pressure waveform that mimics cycles of inhalation and exhalation akin to spontaneous breathing.

In other embodiments, the system is operable to receive a user input 157 to the controller 150 selecting the form of respiratory support that is required to be provided by system 100. The controller then controls the inflation source 155 to achieve inflation or deflation of the inflatable body 125 to a degree of inflation that is sufficient for provision of the selected form of respiratory support. This may involve deflating the inflatable body 125 to a degree of inflation sufficient to achieve non-sealing engagement with the patient's airway for the provision of respiratory support that is independent of spontaneous or non-spontaneous (i.e. artificially ventilated) breathing cycles of the patient, and inflating the inflatable body to achieve sealing engagement with the patient's airway for the provision of a cyclic flow of respiratory gas intended to mimic cycles of inhalation and exhalation akin to spontaneous breathing.

In some embodiments, controller 150 is configurable to determine the presence or absence of spontaneous breathing by patient 300 upon detection of one or more of: a parameter associated with a gas flow to and/or from a patient's airway (e.g. concentration of a target gas e.g. O₂ or CO₂, or flow rate), movement of a patient's chest and a user input confirming there is no observable spontaneous breathing by the patient.

In embodiments where controller 150 receives sensor input that is indicative of either presence or absence of spontaneous breathing by patient 300, the controller may generate an alert to the user via output means 153 to indicate e.g. that there has been a change in breathing status. Alternatively/additionally, the controller may generate an alert to the user via output means 153 to indicate e.g. that there has been a change in the respiratory support provided to the patient. The alert may include an audible sound emitted from a loudspeaker associated with the controller 150 and/or a visible alert presented on a display associated with the controller.

Respiratory Support with Targeted Expiratory Flow Rate

In another embodiment, system 100 is configured for providing respiratory support to a patient 300 during a medical procedure, wherein the respiratory support is controlled to achieve a target expiratory flow rate. System 100 comprises flow source 110 which is configurable to provide a flow of gas for delivery to the patient 300 via an invasive patient interface 120. Flow source 110 is under control of controller 150. System 100 is couplable with or includes an invasive patient interface 120 having a sealable member 125 and is configurable to form a sealing engagement with the patient's airway, or to form a non-sealing arrangement of the invasive patient interface within the patient's airway.

In the non-sealing arrangement in which sealable member (typically an inflatable body or cuff) 125 is not in sealing engagement with airway 310, an exit flow path 270A is created between an external wall of the invasive patient interface 120 and the patient's airway, for flow of gases exiting from the patient's airway. Controller 150 may control the flow source 110 to achieve a target flow rate out of the exit flow path 270A which is referred to as expiratory flow rate. In some embodiments, the target flow rate out of the exit flow path 270A is at least about 0.5 LPM, preferably at least about 5 LPM and in some embodiments at least about 20 LPM.

In some embodiments, the exit flow path 270A has a non-zero cross-sectional area at the sealable member. A larger cross-sectional area may reduce resistance to flow of gases flowing through exit flow path 270A and out of the patient's airway which in turn would allow more flow and increase the flow rate through the invasive patient interface which in turn increases the velocity of gases exiting the invasive patient interface thereby increasing CO₂ clearance. In these embodiments, the exit flow path 270A may provide a path of lower resistance than inside the invasive patient interface 120 for gases at the proximal end of the invasive patient interface thereby aiding in removal of CO₂ and other gases from deep in the patient's airway. Alternatively/additionally, the exit flow path 270A which surrounds the invasive patient interface in the patient's airway comprises, at the location of the sealable member 125, a distance between the sealable member 125 and the airway wall. The required distance may correspond to a non-zero percentage of the patient's airway radius such as e.g. at least about 1%, preferably at least about 2%, or at least about 3% or at least about 4% or at least about 5%. In some embodiments the required distance may correspond to more than 5% of the airway radius, such as e.g. about 10% or more.

Controller 150 may receive inputs indicative of flow rate in exit flow path 270A and either control directly or provide guidance to a user for deflation of an inflatable body comprising the sealable member 125 to achieve the target flow rate through the exit flow path. For example, upon receiving an input indicative of low flow rate (e.g. 0 LPM or about 0.5 LPM) in the exit flow path 270A, controller 150 may control or provide guidance to further deflate the inflatable body 125. When the indicative flow rate reaches about the flow rate delivered into the invasive patient interface, controller 150 may control or provide guidance to cease deflation and commence or modify provision of respiratory support. In some embodiments, controller 150 may control or provide guidance for inflation/deflation of the inflatable body 125 by reference to an algorithm or a look up table stored in memory (locally or remotely) that is readable by the controller and which correlates e.g. fluid inflation volume to be provided by the inflation source, with a desired diameter or degree of inflation of the inflatable body.

In some embodiments, controller 150 may use inputs indicative of flow rate in exit flow path 270A to adjust control of flow source 110. For example, upon receiving an input indicative of a low flow rate which is lower than a target flow rate (e.g. a low flow rate may be 0 LPM or in some cases up to about 0.5 LPM) in exit flow path 270A, controller 150 may operate to increase the rate of flow provided by flow source 110 or provide guidance via output means 153 to manually adjust output of flow source 110 to increase the rate of flow provided.

In some embodiments, controller 150 may use inputs indicative of pressure in the patient's airway to adjust control of flow source 110 to achieve a predetermined patient airway pressure. For example, upon receiving an input indicative of airway pressure below a predetermined airway pressure, controller 150 may adjust control or provide guidance via output means 153 to adjust control or provide guidance via output means 153, to adjust control of flow source 110 to increase the rate of flow provided. Conversely, upon receiving an input indicative of airway pressure above a predetermined airway pressure, controller 150 may adjust control or provide guidance via output means 153 to adjust control of flow source 110 to decrease the rate of flow provided. The predetermined airway pressure may be about 2 cmH₂O, or about 2 cmH₂O to about 20 cmH₂O, or about 2 cmH₂O to about 10cmH₂O, or about 2 cmH₂O and about 5 cmH₂O, or about 5 cmH₂O to about 10 cmH₂O.

Controller 150 may receive inputs comprising signals generated by one or more of an exiting gases flow sensor, a pressure sensor associated with an inflatable body comprising the sealable member, an airway pressure sensor, a gas flow velocity sensor and a user input device as shown schematically in FIG. 10. These inputs may be used by controller 150 to determine when the sealable member 125 is in a non-sealing arrangement within the patient's airway, upon which controller controls or provides guidance to control flow source 110 to provide a substantially continuous flow of gas to the invasive patient interface.

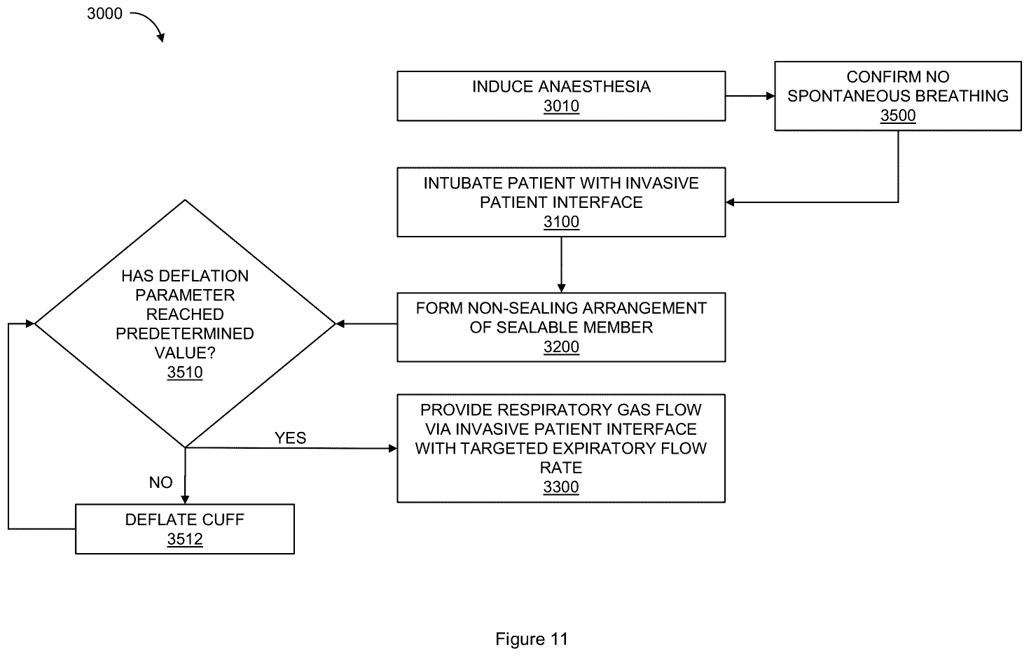

FIG. 11 is a flow chart showing in schematic form steps in a method 3000 for providing respiratory support to a patient during a medical procedure in which an objective is to achieve a target expiratory flow rate. In a step 3100 the patient is intubated with an invasive patient interface having a sealable member operable to form a sealing engagement with the patient's airway. In a step 3200 and while the patient is intubated, the sealable member (typically an inflatable balloon or cuff) is operated (e.g. deflated) to form a non-sealing arrangement of the invasive patient interface within with the patient's airway. A flow of respiratory gas is provided to the patient via the invasive patient interface in step 3300. As discussed in relation to the system 1000 configured for providing respiratory support which is controlled to achieve a target expiratory flow rate, the non-sealing arrangement creates an exit flow path 270A between an external wall of the invasive patient interface and the patient's airway for flow of gases exiting from the patient's airway. According to method 3000, the flow of respiratory gas provided is controlled to achieve a target respiratory support parameter which is associated with flows of gases exiting the patient's airway via the exit flow path 270A.

In some embodiments, the target respiratory support parameter is a target flow rate and in some embodiments may be at least about 0.5 LPM, preferably at least about 5 LPM and in some embodiments at least about 20 LPM.

In some embodiments, step 3200 involves deflating an inflatable body or cuff comprising the sealable member 125 until exit flow path 270A has a non-zero cross-sectional area at the sealable member e.g. its bulkiest part. A larger cross-sectional area may reduce resistance to flow of gases flowing through exit flow path 270A which in turn increases the velocity of gases exiting the invasive patient interface thereby increasing CO₂ clearance. Alternatively/additionally step 3200 involves deflating the cuff 125 until exit flow path 270A surrounding the invasive patient interface 120 in the patient's airway comprises a distance between the sealable member and the airway wall. It is to be understood that exit flow path 270A may surround the invasive patient interface 120 either entirely or partially, the latter encompassing scenarios where the cuff and/or part of the invasive patient interface are arranged along the airway wall. The required distance may correspond to a non-zero percentage of the patient's airway radius such as e.g. at least about 1%, preferably at least about 2%, or at least about 3% or at least about 4% or at least about 5%. In some embodiments the required distance may correspond to more than 5% of the airway radius, such as e.g. about 10% or more.

The substantially continuous flow provided in step 3300 may be a unidirectional or positive net flow towards the patient which in some cases may be time-varying (e.g. oscillatory). Furthermore, the selected flow rate for the flow source 110 may be a fixed flow rate, which may be variable upon input to the controller 150 to vary the rate. The fixed flow rate may be independent of the respiratory cycle of the patient 300. The flow rate may be in a range of 0 LPM to about 150 LPM, preferably about 20 LPM to about 70 LPM and in some embodiments, preferably at least about 40 LPM.

In preferred embodiments, the respiratory support according to method 3000 is provided during absence of spontaneous breathing, to ensure substantial stillness of the respiratory system during the medical procedure. This is particularly important for medical procedures requiring substantial stillness of the abdomen and/or thorax as is often the case. Thus method 3000 may include in an optional step 3500, detecting absence of spontaneous breathing by the patient prior to intubating the patient in step 3100.

In some embodiments it may be desirable to determine that the cuff 125 has been deflated in the non-sealing engagement to a degree that provides sufficient gases outflow within the patient's airway 310, around (at least part of) the invasive patient interface 120 and cuff 125, to form the first exit flow path 270A. Thus, it may be desirable to monitor a parameter indicative of a degree of inflation of cuff 125 in step 3510 to ascertain if the non-sealing arrangement has been established. If the monitored parameter indicates that the non-sealing arrangement has not yet been reached, cuff deflation continues at 3512. If the parameter indicates that the non-sealing arrangement has been reached, deflation ceases and a flow of respiratory gas is provided via the invasive patient interface 120 at step 3300. A clinician or other attendant may determine if the deflation parameter has been met by inspection or observation. In some embodiments, deflation parameter detection may be instrumented using one or more sensors as described in the context of method 1000 which is explicitly stated to be relevant to the method 3000.

Typically, prior to step 3100, patient 300 is induced into a state of general anaesthesia in a step 3010 so that the patient is no longer spontaneously breathing. Following delivery of respiratory gas according to step 3300, the clinician or other operator may cause the cuff to be inflated. Alternatively, the clinician or other operator may provide an input to the controller 150 that the form of respiratory support is to be switched, and the controller will respond by causing inflation of the cuff. Both will cause switching of the respiratory support upon detection by the controller 150 (or observation by a user who provides an input to the controller) that the cuff is in the inflated condition, after which the respiratory support becomes dependent on an inspiratory and expiratory intended breathing cycle of the patient. Subsequent changes in cuff state (i.e. deflation/inflation) and switching between modes of respiratory support may follow as may selected by a clinician or other operator to provide the necessary respiratory support to the patient.

FIGS. 12a to 12h represent in illustrative form, steps in a medical procedure involving methods and systems of the present disclosure. Initially, patient 300 is induced to a state of anaesthesia, e.g. by introducing a drug such as propofol (FIG. 12a) or pentothal into the patient's circulation by injection or intravenous line, or by delivering anaesthetic gases which are inhaled through a mask placed over the patient's nose and mouth. The patient is intubated as previously discussed in relation to FIG. 3 and the sealable member 125 is inflated to form a sealed engagement with the patient's airway as discussed in relation to FIG. 5. The patient receives a flow of gas through the sealed patient interface 120 from a ventilating flow source 112 (FIG. 12b) and the patient is ventilated through the sealed patient interface as represented in FIG. 12c.

In the event that a clinician wishes to commence a procedure that may require patient stillness—specifically respiratory stillness or stillness of the thorax and/or abdomen—the ventilating flow source 112 is paused as represented in FIG. 12d and decoupled from the patient interface 120 as represented in FIG. 12e (although this decoupling may not be required e.g. if a single flow source provides the various forms of respiratory support as required for the procedure). The sealable member 125 of invasive patient interface 120 is then deflated to form a non-sealing arrangement within the patient's airway 310 as discussed in relation to FIG. 4, opening exit flow path 270A which also provides for exit of gases through the patient's nares as well as their mouth. A flow source 110 providing a substantially continuous flow of respiratory gas is coupled with patient interface 120 via humidifier 140 and conduits 160, 180. A controller such as rotary switch 111 or electronic controller 150 is operated to commence provision of substantially continuous flow respiratory support to patient 300 through patient interface 120. While continuous flow respiratory support is being provided to the patient, CO₂ is cleared from the proximal tip of the invasive patient interface 120 and the patient's airways and exits via exit flow path 270A. Provision of CO₂ clearance in this manner extends the safe duration of respiratory support provided to the patient in this state when the patient is not being mechanically ventilated. This enables the clinician to safely and confidently undertake the procedure requiring respiratory stillness.

At the end of the desired period of respiratory stillness, or if a short period of mechanical ventilation is required, the continuous flow respiratory support is ended by operating controller 150/rotary switch 111 to reduce flow to 0 LPM and the patient interface 120 is decoupled from the wye piece connector 185A that provides fluid communication with flow source 110, as illustrated in FIG. 12f. The inflation means 155 is operated to re-inflate the cuff 125 to form sealing engagement within the patient's airway as shown in, and the ventilating flow source 112 is reconnected via e.g. wye piece connector 185B and ventilation of the patient's lungs via invasive patient interface 120 recommences as illustrated in FIG. 12h. Switching between respiratory supports in this way can be performed as many times as is required to achieve desired objectives for the medical procedure. Furthermore, inflation of cuff 125 may be varied to alter the exit flow path 270A to achieve objectives such as increasing or decreasing the resistance to flow in the invasive patient interface, patient's airway and/or exit flow path 270A. This may be desirable in circumstances where e.g. a clinician wishes to prioritise airway pressure over CO₂ clearance, in which case the clinician would increase the amount of cuff inflation to reduce the size of exit flow path 270A thereby increasing patient pressure.

Embodiments of the present disclosure provide novel approaches to the provision of respiratory support to a patient, who may be apnoeic (i.e. not spontaneously breathing), which may improve CO₂ clearance and/or oxygenation of the patient in comparison with previously known practices. This in turn extends the safe duration for which the patient may remain off mechanical ventilation and in “respiratory stillness”. This has particular utility where a medical procedure is best undertaken when there is little or no movement of the lungs and/or thorax and/or abdomen.

In preferred embodiments, the respiratory support during respiratory stillness is achieved by provision of a substantially continuous flow of gas delivered through an invasive patient interface which is located in the patient's airway in a non-sealing arrangement. That is, an inflatable cuff or balloon which is normally inflated for provision of e.g. artificial ventilation or other respiratory support requiring a sealed flow system is deflated sufficiently to provide a gas flow path around the invasive patient interface for exit of gases from the airway. Provision of substantially continuous flow respiratory support, deep in the patient's airway, in combination with an exit flow path that provides for removal of expired and unexchanged gases, improves CO₂ clearance. CO₂ clearance can be further improved by controlling the airway pressure and/or flow velocity delivered at a target location deep in the patient's airway, such as near the carina or in the deeper in one or more bronchi and/or bronchioles, as disclosed herein.

Furthermore, embodiments disclosed herein provide a mechanism by which a clinician or other attendant can switch between different forms of respiratory support. For example, switching between mechanical ventilation (with the cuff inflated) and continuous flow respiratory support (with the cuff deflated). Switching from continuous flow respiratory support may be desirable during a medical procedure to e.g. provide one or more cycles of ventilation, provide pressure, deliver volatile agents to the patient to extend duration of anaesthesia, and reduce risk of gastric aspiration after longer periods of intubation. If a further period of respiratory stillness is required, the cuff can be deflated and the support can be switched back to the substantially continuous form. At conclusion of the medical procedure requiring respiratory stillness, the respiratory support can be switched back to e.g. machine ventilation with the cuff inflated. Where flows corresponding to the respective forms of respiratory support originate from a common flow source, a controller may control one or both of inflation/deflation of the cuff and the flows provided by the flow source. Where flows corresponding to the respective forms of respiratory support originate from different flow sources, switching may also involve coupling and decoupling an inspiratory conduit associated with each flow source with the patient interface (either directly or via an adapter and/or connector) as necessary for provision of the required respiratory support. Switching between forms of respiratory support may be performed as many times as is necessary during the medical procedure.

It is to be understood that various modifications, additions and/or alternatives may be made to the parts previously described without departing from the ambit of the present invention as defined in the claims appended hereto.

The invention may also be said broadly to consist in the parts, elements and features referred to or indicated in the specification of the application, individually or collectively, in any or all combinations of two or more of said parts, elements or features. Where, in the foregoing description reference has been made to integers or components having known equivalents thereof, those integers are herein incorporated as if individually set forth. Similarly, where in the foregoing description reference has been made to features or elements of a particular aspect or embodiment, it is to be understood that those features or elements are herein incorporated as if expressly disclosed in combination with other aspects or embodiments for which a skilled addressee would appreciate those features or elements to be compatible.

Where any or all of the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that the following claims are provided by way of example only, and are not intended to limit the scope of what may be claimed in future. Features may be added to or omitted from the claims at a later date so as to further define or re-define the invention or inventions.

1. A method for providing respiratory support to a patient, the method comprising the steps of:
intubating the patient with an invasive patient interface having a sealable member operable to form a sealing engagement with the patient's airway, wherein while the patient is intubated
the sealable member forms a non-sealing arrangement of the invasive patient interface within the patient's airway; and
providing a flow of respiratory gas to the patient via the invasive patient interface;
wherein the respiratory support is provided during absence of spontaneous breathing.

2. The method according to claim 1, wherein the respiratory support increases CO₂ clearance in the patient.

3. The method according to claim 1, wherein the non-sealing arrangement provides a first exit flow path between an external wall of the invasive patient interface and the patient's airway for out flow of gases from the patient's airway.

4. The method according to claim 3, wherein gases in the first exit flow path have a first predetermined flow rate of at least about 0.5 LPM.

5. (canceled)

6. The method according to claim 3, wherein the invasive patient interface provides a second exit flow path for out flow of gases from the patient's airway, in addition to providing a flow of respiratory gas to the patient.

7. The method according to claim 6, wherein gases in the second exit flow path have a second predetermined flow rate that is less than the first predetermined flow rate.

8. (canceled)

9. The method according to claim 1, wherein the sealable member comprises an inflatable body, and wherein the sealable member is operable to form the non-sealing arrangement by deflating the inflatable body.

10. (canceled)

11. (canceled)

12. (canceled)

13. The method according to claim 9, wherein, when deflated, the inflatable body occupies a cross sectional area corresponding to less than 100% occlusion of the region between the invasive patient interface and the patient's airway.

14. The method according to claim 9, further comprising monitoring a parameter indicative of degree of deflation of the inflatable body during deflation of the inflatable body, and ceasing deflation when the parameter reaches a predetermined value.

15. The method according to claim 14, wherein the parameter is selected one of:
(i) pressure in the inflatable body;
(ii) expiratory flow around the invasive patient interface; or
(iii) expiratory flow within the invasive patient interface.

16. The method according to claim 1, further comprising, prior to intubating the patient, detecting absence of spontaneous breathing by the patient.

17. (canceled)

18. (canceled)

19. The method according to claim 1, wherein the respiratory support is provided in the absence of displacement of one or more of the patient's diaphragm, abdomen, or thorax.

20. The method according to claim 1, wherein the respiratory gas is provided at a flow rate of at least about 40 LPM.

21. (canceled)

22. (canceled)

23. (canceled)

24. The method according to claim 1, wherein the respiratory gas is provided at a predetermined velocity of at least 5 m/s.

25. (canceled)

26. (canceled)

27. The method according to claim 1, wherein the respiratory support provided via the invasive patient interface achieves a predetermined airway pressure of about 2 cmH₂O to about 20.

28. (canceled)

29. The method according to any one of the preceding claims, wherein the invasive patient interface is one of:
(i) an endotracheal tube;
(ii) a laryngeal mask airway (LMA); or
(iii) a tracheostomy tube.

30. The method according to claim 1, wherein the sealable member comprises an inflatable cuff located toward a proximal end of the invasive patient interface and which is inflatable to form the sealing engagement with the patient's airway.

31. The method according to claim 30, wherein the non-sealing engagement is achieved when the inflatable cuff is inflated to an inflation pressure of less than about 30 cmH₂O, less than about 25 cmH₂O, or less than about 20 cmH₂O.

32. The method according to claim 30, further comprising using a pressure sensor to monitor an inflation pressure of the inflatable cuff.

33. (canceled)

34. (canceled)

35. (canceled)

36. (canceled)

37. The method according to claim 1, further comprising, prior to forming the non-sealing arrangement, inducing the patient into a state of general anaesthesia.

38. (canceled)

39. The method of claim 1, further comprising operating the sealable member to form a non-sealing arrangement of the invasive patient interface within the patient's airway.

40. The method according to claim 1, wherein the sealable member comprises an inflatable body, and wherein operating the sealable member is operable to form a sealing arrangement by inflating the inflatable body.
